(19) United States
(12) Patent Application Publication
(10) Pub. No.: US 2021/0378367 A1
(43) Pub. Date: Dec. 9, 2021

(54) COMPOSITE ELASTIC MATERIAL INCLUDING STRUCTURED FILM AND PROCESS FOR MAKING THE SAME

(71) Applicant: 3M INNOVATIVE PROPERTIES COMPANY, St. Paul, MN (US)

(72) Inventors: Thomas J. Gilbert, St. Paul, MN (US); Todd L. Nelson, Eau Claire, WI (US); Neelakandan Chandrasekaran, Plymouth, MN (US); Mark A. Peltier, Forest Lake, MN (US); Scott M. Niemi, St. Paul, MN (US); Stanley Rendon, Eagan, MN (US)

(21) Appl. No.: 17/283,070

(22) PCT Filed: Oct. 8, 2019
(86) PCT No.: PCT/IB2019/058557

§ 371 Date: Apr. 6, 2021

Related U.S. Application Data
(60) Provisional application No. 62/742,734, filed on Oct. 8, 2018.

(51) Int. Cl. A44B 18/00; A61F 13/62

(52) U.S. Cl. CPC A44B 18/0015; A61F 13/625; B29C 66/7294; A44B 18/0069; A44B 18/0061

(57) ABSTRACT

The composite elastic material (22) includes an elastic layer (4) and a structured film layer (15) having first and second opposing surfaces, with the second surface bonded to the elastic layer (4). The first surface of the structured film layer (15) has upstanding male fastening elements. The structured film layer (15) is gathered such that the upstanding male fastening elements point in multiple directions. The composite elastic material (22) can also be called a stretch-bonded laminate, which include an elastic layer (4) stretch-bonded to a second surface of a structured film layer (15). A first surface of the structured film layer (15), opposite the second surface, has upstanding male fastening elements. A process for making the composite elastic material (22) is also described. An absorbent article including the composite elastic material (22) is also described.

### CROSS-REFERENCE TO RELATED APPLICATION

This application claims priority to U.S. Provisional Application No. 62/742,734, filed Oct. 8, 2018, the disclosure of which is incorporated by reference in its entirety herein.

### BACKGROUND

Articles with one or more structured surfaces are useful in a variety of applications (e.g., abrasive discs, assembly of automobile parts, and disposable absorbent articles). The articles may be provided as films that exhibit, for example, increased surface area, mechanical fastening structures, or optical properties.

Mechanical fasteners, which are also called hook and loop fasteners, typically include a plurality of closely spaced upstanding projections with loop-engaging heads useful as hook (i.e., male fastening element) members, and loop (female fastening element) members typically include a plurality of woven, nonwoven, or knitted loops. Mechanical fasteners are useful for providing releasable attachment in numerous applications. For example, mechanical fasteners are widely used in wearable disposable absorbent articles to fasten such articles around the body of a person. In typical configurations, a hook strip or patch on a fastening tab attached to the rear waist portion of a diaper or incontinence garment, for example, can fasten to a landing zone of loop material on the front waist region, or the hook strip or patch can fasten to the backsheet (e.g., nonwoven backsheet) of the diaper or incontinence garment in the front waist region.

It can be useful for a fastening tab including to have elasticity. For example, in absorbent articles, fit, comfort, and design versatility may be improved by using elastic fastening tabs. Hook fasteners are typically made by forming hook elements on a film backing made from inelastic materials to achieve better engagement and shear strength when engaged with corresponding loop materials. However, a rigid nonelastic fastener imparts a dead zone wherein it is attached to an elastic substrate, for example. This dead zone causes a loss of extension on an elastically extensible waist margin of the diaper and may have a deleterious effect on the fit of the diaper to the wearer. In some cases, separated narrow strips of hook fasteners or even individual hooks have been applied to or formed on an elastic substrate to improve the extensibility of the region including a hook fastener. See, for example, U.S. Pat. No. 6,080,347 (Goulait); U.S. Pat. No. 6,146,369 (Hartmann); U.S. Pat. No. 6,419,667 (Avalon); U.S. Pat. No. 6,489,003 (Levitt); U.S. Pat. No. 7,048,818 (Krantz); U.S. Pat. No. 7,125,400 (Igaue); and U.S. Pat. No. 7,223,314 (Provost).

### SUMMARY

The present disclosure provides a laminate with an elastic layer where the mechanical fastening portion is also stretchable. Gathers in a structured film layer having upstanding male fastening elements allow it to extend when the elastic layer is stretched. The elastic composite material disclosed herein exhibits elastic properties as described herein in the area where the structured film layer and the elastic layer overlap.

In one aspect, the present disclosure provides a composite elastic material that includes an elastic layer and a structured film layer having first and second opposing surfaces, with the second surface bonded to the elastic layer. The first surface of the structured film layer has upstanding male fastening elements. The structured film layer is gathered such that the upstanding male fastening elements point in multiple directions. It should be understood that the structured film layer is gathered when the elastic layer is in a relaxed state, with no tension applied.

In another aspect, the present disclosure includes process for making the composite elastic material disclosed herein. The process includes stretching the elastic layer in a first direction, bonding the second surface of the structured film layer to the elastic layer while the elastic layer is stretched, and allowing the elastic layer to relax and the structured film layer to gather to form the composite elastic material.

In another aspect, the present disclosure provides a stretch-bonded laminate including an elastic layer stretch-bonded to a second surface of a structured film layer. A first surface of the structured film layer, opposite the second surface, has upstanding male fastening elements.

In another aspect, the present disclosure includes process for making the stretch-bonded laminate disclosed herein. The process includes stretching the elastic layer in a first direction and bonding the second surface of the structured film layer to the elastic layer while the elastic layer is stretched.

In another aspect, the present disclosure provides an absorbent article including the composite elastic material and/or stretch-bonded laminate described herein.

As used herein, the recitation of numerical ranges by endpoints includes all numbers subsumed within that range (e.g. 1 to 5 includes 1, 1.5, 2, 2.75, 3, 3.8, 4, and 5, and the like).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

For the following defined terms, these definitions shall be applied for the entire Specification, including the claims, unless a different definition is provided in the claims or elsewhere in the Specification based upon a specific reference to a modification of a following defined term:

The words “a”, “an”, and “the” are used interchangeably with “at least one” to mean one or more of the elements being described.

The phrase “comprises at least one of” followed by a list refers to comprising any one of the items in the list and any combination of two or more items in the list. The phrase “at least one of” followed by a list refers to any one of the items in the list or any combination of two or more items in the list.

The term “nonwoven” refers to a material having a structure of individual fibers or threads that are interlaid but not in an identifiable manner such as in a knitted fabric.

The term “layer” refers to any material or combination of materials on or overlaying a substrate.

The term “acrylic” refers to compositions of matter which have an acrylic or methacrylic moiety.

Words of orientation such as “atop, “on,” “covering.” “uppermost,” “overlaying,” “underlying” and the like for describing the location of various layers, refer to the relative position of a layer with respect to a horizontally-disposed, upwardly-facing substrate. It is not intended that the substrate, layers or articles encompassing the substrate and layers, should have any particular orientation in space during or after manufacture.

The term “separated by” to describe the position of a layer with respect to another layer and the substrate, or two other layers, means that the described layer is between, but not necessarily contiguous with, the other layer(s) and/or substrate.

The term “(co)polymer” or “(co)polymeric” includes homopolymers and copolymers, as well as homopolymers or copolymers that may be formed in a miscible blend, e.g., by coextrusion or by reaction, including, e.g., transesterification. The term “copolymer” includes random, block, graft, and star copolymers.

The term “structured film” refers to a film with other than a planar or smooth surface.

The term “in-line,” as used herein, means that the steps are completed without the thermoplastic layer being rolled up on itself. The steps may be completed sequentially with or without additional steps in-between. For clarification, the thermoplastic layer may be supplied in rolled form and the finished laminate may be rolled up on itself.

The term “machine direction” (MD) as used herein denotes the direction of a running, continuous web. In a roll, for example, comprising an elastic layer and a structured film layer, the machine direction corresponds to the longitudinal direction of the roll. Accordingly, the terms machine direction and longitudinal direction may be used herein interchangeably. The term “cross-direction” (CD) as used herein denotes the direction that is essentially perpendicular to the machine direction.

The term “discontinuous” refers to bonding that is not continuous in at least one direction. Bonding may appear continuous in one direction and still be discontinuous if it is not continuous in another direction.

The term “stretch-bonded laminate” refers to a composite material having at least two layers in which one layer is a gatherable layer and the other layer is an elastic layer. The layers are joined together when the elastic layer is extended from its original condition so that upon relaxing the layers, the gatherable layer is gathered. Such a composite elastic material may be stretched to the extent that the nonelastic material gathered between the bond locations allows the elastic material to elongate. The composite elastic material disclosed herein is a stretch-bonded laminate, and the term “composite elastic material” can be substituted with the term “stretch-bonded laminate” in any of the embodiments disclosed herein.

The term “elastic” refers to any material (such as a film that is 0.002 mm to 0.5 mm thick) that exhibits recovery from stretching or deformation. In some embodiments, a material may be considered to be elastic if, upon application of a stretching force, it can be stretched to a length that is at least about 25 (in some embodiments, 50) percent larger than its initial length at room temperature and can recover at least 40, 50, 60, 70, 80, or 90 percent of its elongation upon release of the stretching force.

As used herein, the term “recover” and variations thereof refer to a contraction of a stretched material upon termination of a biasing force following stretching of the material by application of the biasing force.

The term “microporous” refers to having multiple pores that have a largest dimension (in some cases, diameter) of up to 10 micrometers. Pore size is measured by measuring bubble point according to ASTM F-316-80.

Various aspects and advantages of exemplary embodiments of the present disclosure have been summarized. The above Summary is not intended to describe each illustrated embodiment or every implementation of the present disclosure. Further features and advantages are disclosed in the embodiments that follow. The Drawings and the Detailed Description that follow more particularly exemplify certain embodiments using the principles disclosed herein.

### BRIEF DESCRIPTION OF THE DRAWINGS

The disclosure may be more completely understood in consideration of the following detailed description of various embodiments of the disclosure in connection with the accompanying figures, in which:

FIG. 1 is a schematic view of an embodiment of the process of the present disclosure.

FIG. 2 is a plan view of an embodiment of the composite elastic material of the present disclosure, shown with parts broken away and in a stretched condition.

FIG. 2A is an enlarged section view along a portion of line 2A-2A of FIG. 2 but with the composite elastic material in a relaxed condition relative to its condition in FIG. 2.

FIG. 3 is an isometric view of an embodiment of the composite elastic material of the present disclosure, in which the structured film laver is a microporous film including a beta-nucleating agent.

FIG. 4 is a perspective view of an embodiment of an absorbent article including the composite elastic material according to the present disclosure.

FIG. 5 is a perspective view of another embodiment of an absorbent article including the composite elastic material according to the present disclosure.

While the above-identified drawings, which may not be drawn to scale, set forth various embodiments of the present disclosure, other embodiments are also contemplated, as noted in the Detailed Description. In all cases, this disclosure describes the presently disclosed invention by way of representation of exemplary embodiments and not by express limitations. It should be understood that numerous other modifications and embodiments can be devised by those skilled in the art, which fall within the scope and spirit of this disclosure.

### DETAILED DESCRIPTION

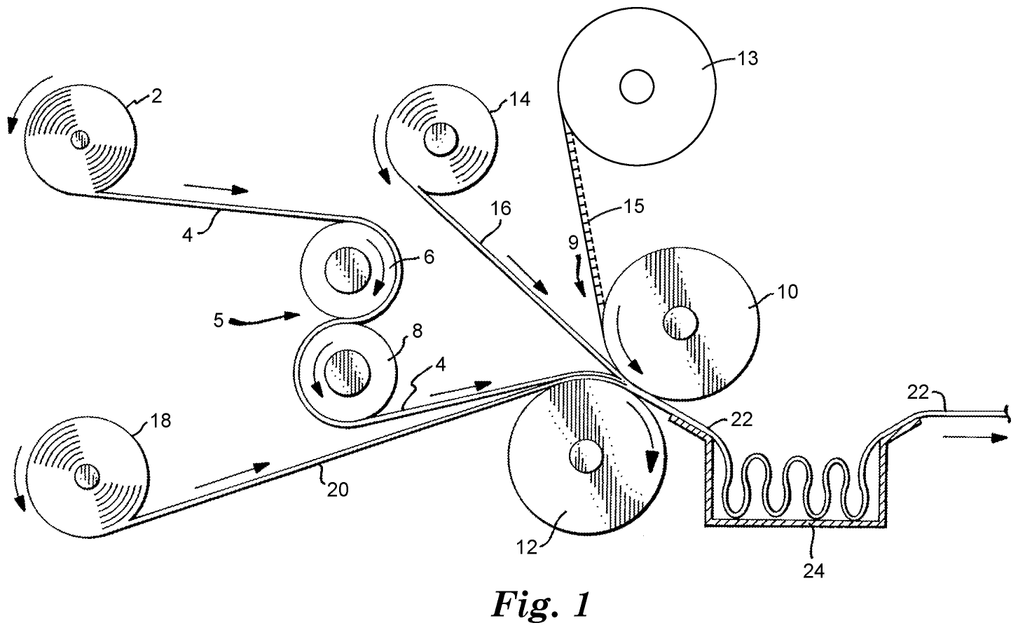

Referring now to FIG. 1 of the drawings, a process for making an embodiment of the composite elastic material of the present disclosure is schematically illustrated. An elastic web 4 is unwound from a supply roll 2 and, traveling in the direction indicated by the arrows associated therewith, passes through the nip of reverse S roll arrangement 5, including stacked rollers 6, 8. From reverse S roll arrangement 5, web 4 passes into the pressure nip of a bonder roll arrangement 9, which includes a patterned calender roller 10 and a smooth anvil roller 12. A structured film web 15 having upstanding male fastening elements is unwound from supply roll 13. In the illustrated embodiment, first fibrous web 16 is unwound from a supply roll 14, and a second fibrous web 20 is unrolled from a supply roll 18. The structured film web 15, first fibrous web 16, and second fibrous web 20 travel in the direction indicated by the illustrated arrows as supply rolls 13, 14, and 18 rotate in the directions indicated by the respective arrows. The elastic web 4 is stretched to a desired percent elongation between S roll arrangement 5 and the pressure nip of bonder roll arrangement 9, which are set at different speeds. The peripheral linear speed of the rollers of S roll arrangement 5 is controlled to be less than the peripheral linear speed of the rollers of bonder roll arrangement 9. Web is maintained in such elongated condition during heat-bonding of the webs 15, 16, and 20 to the web 4 in bonder roll arrangement 9. One or both of patterned calender roller 10 and smooth anvil roller 12 may be heated and the pressure between these two rollers may be adjusted by a variety of means to provide the desired temperature and bonding pressure to bond the webs 15, 16, and 20 to the web 4 and form a composite elastic material 22. A variety of conventional drive means and other conventional devices may be useful in conjunction with the apparatus of FIG. 1, but for purposes of clarity, they have not been illustrated in the schematic view of FIG. 1. In the embodiment illustrated in FIG. 1, fibrous webs are bond to each of the two opposite sides of a stretched elastic web, and the structured film web 15 is bonded to the stretched elastic web with one of the fibrous webs in between. In other embodiments, one of the fibrous webs is absent or both of the fibrous webs are absent.

The process for making the composite elastic material of the present disclosure includes stretching the elastic layer in a first direction. In some embodiments, the first direction is the machine direction. While in the illustrated embodiment, monoaxial stretching in the machine direction is performed by propelling the elastic web over rolls of increasing speed, other methods of stretching the elastic web are possible. A versatile stretching method that allows for monoaxial, sequential biaxial, and simultaneous biaxial stretching of a web employs a flat film tenter apparatus. Such an apparatus grasps the web using a plurality of clips, grippers, or other film edge-grasping means along opposing edges of the web in such a way that monoaxial, sequential biaxial, or simultaneous biaxial stretching in the desired direction is obtained by propelling the grasping means at varying speeds along divergent rails. Increasing clip speed in the machine direction generally results in machine-direction stretching. In a small-scale process instead of a web process, the elastic layer can be stretched, for example, by hand.

The process for making the composite elastic material of the present disclosure includes bonding the second surface of the structured film layer to the stretched elastic layer. Although FIG. 1 uses calendering to bond the layers of the web laminate, it should be understood that the structured film webs and fibrous webs may be laminated to the elastic web by a variety of processes including calendering, adhesive bonding, bonding with a heated fluid, ultrasonic welding, and combinations thereof.

In some embodiments, the second surface of the structured film layer is bonded to the elastic layer with adhesive. Thus, in some embodiments, the composite elastic material includes an adhesive layer, which may be continuous or discontinuous, between the elastic layer and the structured film layer. Similarly, in some embodiments, the process for making the composite elastic material includes disposing a layer of adhesive, which may be continuous or discontinuous, between the elastic layer and the structured film layer. Suitable adhesives include water-based, solvent-based, pressure-sensitive, and hot-melt adhesives. Pressure sensitive adhesives (PSAs) are known to those of ordinary skill in the art to possess properties including the following: (1) aggressive and permanent tack, (2) adherence with no more than finger pressure, (3) sufficient ability to hold onto an adherend, and (4) sufficient cohesive strength to be cleanly removable from the adherend. Materials that have been found to function well as PSAs are polymers designed and formulated to exhibit the requisite viscoelastic properties resulting in a desired balance of tack, peel adhesion, and shear holding power. Suitable pressure sensitive adhesives include acrylic resin and natural or synthetic rubber-based adhesives and may be hot melt pressure sensitive adhesives. Illustrative rubber based adhesives include styrene-isoprene-styrene, styrene-butadiene-styrene, styrene-ethylene/butylenes-styrene, and styrene-ethylene/propylene-styrene that may optionally contain diblock components such as styrene isoprene and styrene butadiene. Any of these adhesives may be tackified, for example, with a synthetic polyterpene resin. The adhesive may be applied using hot-melt, solvent, or emulsion techniques. Adhesive bonding the second surface of the structured film layer to the elastic layer may be useful, for example, because it generally would not impact the upstanding male fastening elements on the first surface of the structured film layer.

In some embodiments, discontinuous bonding is carried out with an ultrasonic horn and a patterned anvil roll. The ultrasonic horn may be stationary or rotary. Ultrasonics may include vibration frequencies above, at, or below the audible range and would be chosen to efficiently bond the polymers taking into account the complex viscosity of the polymer being bonded. In some embodiments, the upstanding male fastening elements of the structured film layer are positioned toward the patterned anvil roll, and the elastic layer and optionally other fibrous layer is positioned toward the ultrasonic horn. This configuration may be useful, for example, for protecting unbonded upstanding male fastening elements from damage. Although in other embodiments, the upstanding male fastening elements can be positioned away from patterned anvil roll and toward the ultrasonic horn. The depth of the anvil pattern is generally similar to the overall thickness of the structured film layer and elastic film layer. Ultrasonic welding using a stationary horn and a rotating patterned anvil roll is described in U.S. Pat. No. 3,844,869 (Rust Jr.) and U.S. Pat. No. 4,259,399 (Hill). Ultrasonic welding using a rotary horn with a rotating patterned anvil roll is described in U.S. Pat. No. 5,096,532 (Neuwirth, et al.); U.S. Pat. No. 5,110,403 (Ehlert); and U.S. Pat. No. 5,817,199 (Brennecke, et al.). Other ultrasonic welding techniques may also be useful.

The raised areas on the calender roll or anvil roll for bonding at spaced-apart locations are selected to provide a desired bonding pattern. The raised areas may be in one or more regular patterns or may be asymmetric across the roll. For example, there may be a zone on the roll having a particular size, shape, or density of raised areas and another zone on the roll that differs in the size, shape, or density of the raised areas. The roll may be designed such that the portion of the roll that contacts the overlapping area of the elastic layer and the structured film layer provides one pattern or more than one pattern of bond sites. For strips of structured film layers that are smaller in area than the elastic layer and optional other fibrous layers, it is also envisioned that a portion of the roll that contacts the elastic layer and one or more fibrous layers only has a different pattern than the portion of the roll that contacts the overlapping area of the elastic layer, the structured film layer, and optionally other fibrous layers.

In some embodiments, including embodiments in which the elastic layer is a fibrous layer, the structured film layer can be joined to the elastic layer using surface bonding or loft-retaining bonding techniques. The term “surface-bonded” when referring to the bonding of fibrous materials means that parts of fiber surfaces of at least portions of fibers are melt-bonded to the second surface of the structured film layer, in such a manner as to substantially preserve the original (pre-bonded) shape of the second surface of the structured film layer, and to substantially preserve at least some portions of the second surface of the structured film layer in an exposed condition, in the surface-bonded area. Quantitatively, surface-bonded fibers may be distinguished from embedded fibers in that at least about 65% of the surface area of the surface-bonded fiber is visible above the second surface of the structured film layer in the bonded portion of the fiber. Inspection from more than one angle may be necessary to visualize the entirety of the surface area of the fiber. The term “loft-retaining bond” when referring to the bonding of fibrous materials means a bonded fibrous material comprises a loft that is at least 80% of the loft exhibited by the material prior to, or in the absence of, the bonding process. The loft of a fibrous material as used herein is the ratio of the total volume occupied by the web (including fibers as well as interstitial spaces of the material that are not occupied by fibers) to the volume occupied by the material of the fibers alone. If only a portion of a fibrous web has the second surface of the structured film layer bonded thereto, the retained loft can be easily ascertained by comparing the loft of the fibrous web in the bonded area to that of the web in an unbonded area. It may be convenient in some circumstances to compare the loft of the bonded web to that of a sample of the same web before being bonded, for example, if the entirety of fibrous web has the second surface of the structured film layer bonded thereto.

In some embodiments, bonding the second surface of the structured film layer to the stretched elastic layer to form the composite elastic material comprises impinging heated fluid (e.g., ambient air, dehumidified air, nitrogen, an inert gas, or other gas mixture) onto at least one of the structured film layer or the elastic layer. In some embodiments, the heated fluid is heated air. In some embodiments, bonding the second surface of the structured film layer to the stretched elastic layer comprises impinging heated fluid onto a first surface of the elastic web while it is moving and/or impinging heated fluid onto the second surface of the structured film web while it is moving and contacting the first surface of the elastic web with the second surface of the structured film web so that the first surface of the clastic web is melt-bonded (e.g., surface-bonded or bonded with a loft-retaining bond) to the second surface of the structured film web. Impinging heated fluid onto the first surface of the elastic web and impinging heated fluid on the second surface of the structured film web may be carried out sequentially or simultaneously. In some embodiments, the bonding method includes impinging gaseous fluid on the second surface of the structured film web and moving the elastic web through ambient-temperature quiescent air before contacting the first surface of the elastic web with the second surface of the structured film web so that the first surface of the elastic web is melt-bonded to the second surface of the structured film web. Further methods and apparatus for joining a continuous web to a fibrous carrier web using high-temperature impingement fluid are described in U.S. Pat. No. 9,096,960 (Biegler et al.), U.S. Pat. No. 9,126,224 (Biegler et al.), and U.S. Pat. No. 8,956,496 (Biegler et al.).

Sufficient heat and/or pressure upon structured film layer and stretched elastic layer is generally used during calendering, ultrasonic welding, and bonding with heated fluid such that at least a portion of the structured film layer and/or elastic web layer are softened or melted to the extent that they may be bonded. Combinations of any of the above bonding methods may be useful for bonding the elastic layer to the structured film layer.

The process of the present disclosure includes allowing the elastic layer to relax and the structured film layer to gather to form the composite elastic material. In the embodiment illustrated in FIG. 1, composite elastic material 22 emerges from the nip of bonder roll arrangement 9 and passes to a holding box 24 wherein it is maintained in a relaxed (i.e., unstretched) condition for a length of time (e.g., up to one minute, up to about 30 seconds, or in a range from about 3 to 20 seconds) sufficient for elastic web 4 to cool. This brief recovery period in a relaxed condition at room temperature immediately after bonding may be useful in some cases for maintaining the elasticity of the composite elastic material.

In some embodiments, relaxation of the composite elastic material is accomplished by rolls of different speeds. As described above, the rollers of bonder roll arrangement 9 are set at a faster speed rollers of S roll arrangement 5, causing the elastic web 4 to stretch. After composite elastic material 22 emerges from the nip of bonder roll arrangement 9, it can be directed over another roller (not shown) that is set at a slower speed than bonder roll arrangement 9, causing the composite elastic material 22 to relax.

After relaxation composite elastic material 22 can be wound up on a storage roll, not shown. It is also possible to combine the process of making composite elastic material with a downline process of manufacturing an article. For example, the composite elastic material 22 may be maintained in a stretched state after it is withdrawn from the bonder roll arrangement 9 and incorporated into an article in a downline process before allowing the composite elastic material to recover. Any of these relaxation methods may be combined with any of the bonding methods and stretching methods described above.

Conveniently, the structured film web useful in the process illustrated in FIG. 1 can be supplied from an unwind stand. In these embodiments, the process according to the present disclosure includes further comprising unwinding the structured film layer from a roll before bonding it to the elastic layer.

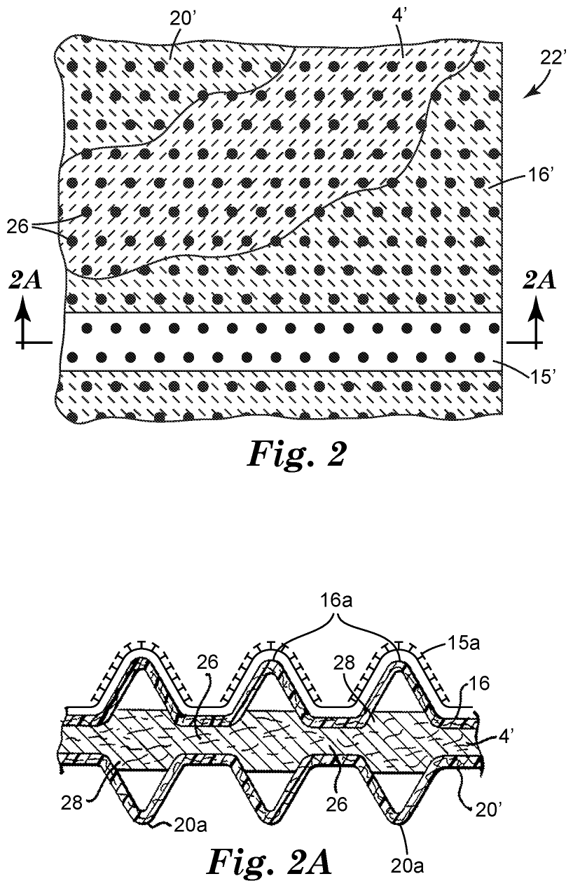

Referring now to FIGS. 2 and 2A, a plan view of an embodiment of the composite elastic material of the present disclosure is illustrated. The composite elastic material 22′ includes a structured film layer 15′, a first fibrous layer 16′, and a second fibrous layer 20′ bonded to elastic layer 4′. In the illustrated embodiment, structured film layer 15′ and first fibrous layer 16′ are bonded to one major surface of the elastic layer 4′, and the second fibrous layer 20′ is bonded to the opposite major surface of the elastic layer 4′. A cross-section of the composite elastic material of the present disclosure is shown in FIG. 2A. The composite elastic material includes gathers 15a, 16a, and 20a formed in layers 15′, 16′, and 20′, respectively. Gathers 15a, 16a, and 20a are not shown in FIG. 2 in order to suggest the appearance of the composite elastic material 22′ in its stretched condition. Gathers 15a, 16a, and 20a are present when the composite material 22′ is in a relaxed condition as shown in FIG. 2A. In some embodiments, including the embodiment illustrated in FIGS. 2 and 2A, the structured film layer 15′, first fibrous layer 16′, and second fibrous layer 20′ are discontinuously bonded to the elastic layer 4′ at spaced-apart locations corresponding to indented areas 26, and gathers 15a, 16a, and 20a form between the spaced-apart locations. In other embodiments, at least the structured film layer 15′ is continuously bonded to the elastic layer 4′, and gathers 15a form in a more random fashion. To form gathers, the structured film layer is generally integral (that is, forming one piece) in the direction of stretch. On the other hand, separate pieces of structured film that are attached to an elastic layer while it is in a relaxed condition can allow the elastic layer to stretch, at least in the areas not bonded to the structured film layer, but would not be gathered when the elastic layer returns to a relaxed condition.

The structured film layer is gathered when tension in the composite elastic material is not being applied. As illustrated in FIG. 2A, this means that the structured film layer is drawn together to form gathers 15a, which may also be understood as puckers, wrinkles, or areas where the structured film layer is at least partially folded back on itself. When tension is applied to the composite elastic material, the gathers can be expanded (in other words, straightened out or unfolded) such that the composite elastic material stretches as far as the relatively inelastic structured film layer allows the elastic material to elongate. In this way, the gathers facilitate elongation of the composite elastic material. When the stretching force applied is less than the failure strength of the structured film layer, the structured film layer can act as a “stop” to prevent further elongation of the elastic material.

Even when upstanding male fastening elements are formed on a backing such that they all point in the same direction, when the structured film layer is gathered, the upstanding male fastening elements point in multiple directions depending on their location on the gathers 15a. For example, on the crest or peak of the gathers 15a, the upstanding posts appear to be perpendicular to the plane defined by the elastic film layer. Closer to the bonded areas corresponding to indented areas 26, the direction of the upstanding male fastening elements is at an oblique angle to the plane defined by the elastic film layer. Multiple angles of the upstanding male fastening elements relative to the plane defined by the elastic film layer may exist between the trough and crest of the gathers. The differences in the orientation of the male fastening elements may provide benefits, for example, in forming strong attachment to loop materials.

The gathers in the composite elastic material result from the elastic layer being bonded to the structured film layer while it is stretched and the tension being subsequently released. Such gathers do not form when the structured film layer is bonded to an elastic layer while the elastic layer is relaxed. Furthermore, when a structured film layer is extrusion laminated to an elastic film or prepared as a multilayer coextruded film with a structured film layer and an elastic layer, wrinkles in the structured film layer are said to form between two adjacent stems after the film is stretched and relaxed (see, e.g., U.S. Pat. No. 6,489,003 (Levitt et al.). In such cases, the stems would not point in multiple directions in relative to the plane of the film.

Referring again to FIGS. 1 and 2, elastic web 4′ has a plurality of indented areas 26 formed therein corresponding to the raised portions of a repeating pattern on the calender roller 10. The indented areas 26 can be formed if sufficient temperature and pressure is maintained in the nip between the calender roller 10 and anvil roller 12. The peripheral portions 28 of the indented areas 26 of the web 4′ illustrated in FIG. 2A can include a resolidified portion of the material which was formerly located in the indented area 26 of elastic web 4′ but melted or softened in the nip. The bond strength between the elastic layer 4′ and first and second fibrous layers 16′ and 20′ may be highest at peripheral portions 28. Indented areas 26 may also be formed when an ultrasonic horn and a pattern roll are used for bonding. In some cases, depending upon the temperature and pressure imposed upon the layers during bonding, material may be forced from the areas of the layers which are compressed by the raised portions of the patterned roller, resulting in a pattern of fine holes in at least one of the elastic layer or structured film layer. Such holes would typically be surrounded by bonded peripheral portions 28 of the elastic layer, structured film layer, and optionally other layers.

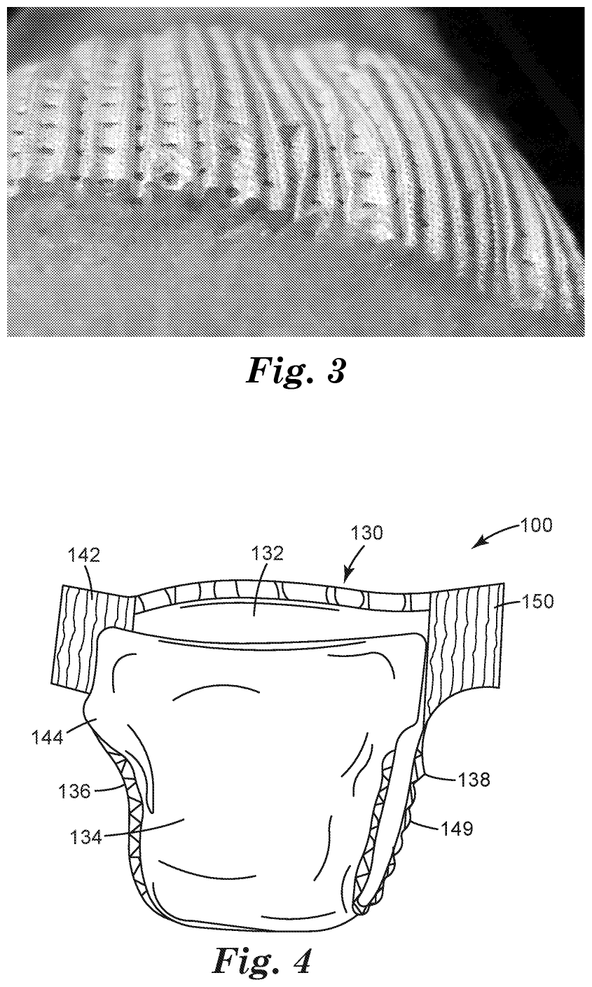

Discontinuously bonding the structured film layer to the elastic layer using at least one of heat, pressure, or ultrasonics using a pattern roller as described above can also destroy the upstanding male fastening elements in the bond sites. In these embodiments, the composite elastic material lacks male fastening elements in indented areas 26 as illustrated in FIG. 2A. The backing of the structured film layer may also be indented at the bond sites as shown in FIG. 3, resulting in reduced film thickness at the bond sites. FIG. 3 illustrates a pattern of circular indentations formed by the patterned roller. Other changes in the structured film layer may also be present at the bond sites. For example, for microporous films, the microporous structure may be collapsed at the bond sites as described in further detail below.

In selecting a set of bond sites for discontinuously bonding the structured film layer to the elastic layer, the strength of the bond of the structured film layer to the elastic layer, the stiffness of the composite elastic material, and the destruction of the male fastening elements on the structured film may all be considered and balanced against each other. For example, a set of bond sites with a high bond area may ensure a strong bond between the elastic layer and the structured film layer but may crush too many male fastening elements, which may affect the performance of the structured film layer, and may increase the stiffness of the composite elastic material to a level that is undesirable. Conversely, a set of bond sites with a low bond area may minimize the effect on male fastening elements of the structured film but decrease the bond strength between layers.

The composite elastic material according to the present disclosure and/or made by the process of the present disclosure may have any desired size, and the individual layers may have any desired size relative to each other. In the embodiments illustrated in FIGS. 2, 2A, and 3, the structured film layer is a strip smaller in at least one dimension than the elastic layer. In other embodiments, the structured film layer may be large enough to cover one major surface of the elastic layer and may be coextensive with the elastic layer. In some embodiments in which the structured film layer and the elastic layer have the same area, the overlapping area is the same as the area of the entire composite elastic material, and the perimeters of the structured film layer and elastic layer are coincident. In other embodiments, depending on the desired use of the composite elastic material, the structured film layer may be larger in at least one dimension than the elastic layer or the layers may be offset; however, the structured film layer will only exhibit elastic properties where it overlays the elastic layer.

In some embodiments, at least two strips of the structured film layer are bonded to the elastic layer. The second strip (and optionally further strips) is also stretch-bonded to the elastic layer and in the same manner as the structured film layer and gathered such that the upstanding male fastening elements point in multiple directions. Strips of the structured film layer may have the same or different size and shape and may be bonded to the elastic layer in any desired configuration relative to each other. In some embodiments, two or more (e.g., three or four) strips of structured film layer are bonded side-by-side to the elastic layer. The two or more strips of structured film may be abutting, or they may be separated by a distance that is usually smaller than the width of each strip (that is, in the direction perpendicular to the longest dimension of the strip of structured film and to the thickness dimension, which is the smallest dimension of the strip of structured film). An example of a suitable configuration of two fastening patches that may be useful for two strips of structured film is described in Int. Pat. Appl. Pub, No. WO 2011/163020 (Hauschildt et al.). The strips are generally longer and integral (i.e., forming one piece) in the direction of stretch. The two or more strips of structured film layer may be the same or different sizes in any of the length, width, or thickness dimension.

The elastic layer in the composite elastic material and process for making the composite elastic material of the present disclosure can be in a variety of forms. For example, the elastic layer can be a fibrous elastic material (e.g., a woven web, nonwoven web, a knitted web, textile, or a combination thereof) or an elastic film (e.g., blown or cast film or multilayer film). In some embodiments, the elastic layer comprises a plurality of elastic strands. While an elastic useful for practicing the present disclosure can be stretched to a length that is at least about 25 (in some embodiments, 50) percent larger than its initial length, typically, the elastic layer is capable of undergoing up to 300% to 1200% elongation at room temperature, and in some embodiments up to 600% to 800% elongation at room temperature. The elastic layer can be made from pure elastomers or blends with an elastomeric phase or content as long as it exhibits elastic behavior as described herein.

Examples of nonwoven webs that may be useful for the elastic layer useful for practicing the present disclosure include spunbond webs, spunlaced webs, airlaid webs, meltblown web, combinations thereof, and combinations of these with other fibers (e.g. staple fibers). The length of the fibers suitable for forming the elastic layer can vary depending on the method used for forming the web. In some embodiments, the elastic layer comprises fibers of effectively endless length. In some embodiments, the elastic layer comprises staple fibers, which may have a length, for example, up to 10 centimeters (cm), in some embodiments, in a range from 1 cm to 8 cm, 0.5 cm to 5 cm, or 0.25 cm to 2.5 cm. In some embodiments, the elastic layer comprises at least one of spunlaid fibers or meltblown fibers. In some embodiments, the fibers of the elastic nonwoven layer have diameters of up to 100 micrometers, in some embodiments, in a range from 1 to 50 micrometers.

Spunlaid nonwovens can be made, for example, by extruding a molten thermoplastic as filaments from a series of fine die orifices in a spinneret. The diameter of the extruded filaments is rapidly reduced under tension by, for example, non-eductive or eductive fluid-drawing or other known mechanisms, such as those described in U.S. Pat. Nos. 4,340,563, 3,692,618, 3,338,992, 3,341,394, 3,276,944, 3,502,538, 3,502,763, and 3,542,615. Nonwoven fabrics made in this manner that are subsequently bonded (e.g., point bonded or continuously bonded) are generally referred to as spunbond nonwovens.

Meltblown nonwovens can be made, for example, by extrusion of thermoplastic polymers from multiple die orifices, which polymer melt streams are immediately attenuated by hot high velocity air or steam along two faces of the die at the location where the polymer exits from the die orifices. The resulting fibers are entangled into a coherent web layer in the resulting turbulent airstream prior to collection on a collecting surface. While meltblown nonwovens have some integrity upon forming due to entanglement, generally, to provide sufficient integrity and strength, meltblown nonwovens are typically further bonded (e.g., point bonded or continuously bonded).

Bonded elastic nonwoven webs useful as the elastic layer in the composite elastic material and process according to the present disclosure are typically bonded (e.g., point bonded or continuously bonded) before being bonded to the structured film layer. Accordingly, the bonded elastic nonwoven can have a bonding pattern distinct from the bonding pattern used for bonding the elastic layer to the structured film layer. Such a distinct bonding pattern can be observed in the areas of the bonded elastic nonwoven that extend beyond the border of the structured film layer or on the surface of the elastic film layer opposite the structured film layer.

Examples of polymers for making elastic fibers, strands, and films (e.g., the core of the multilayer film described below) include thermoplastic elastomers such as ABA block copolymers, polyurethane elastomers, polyolefin elastomers (e.g., metallocene polyolefin elastomers, ethylene/propylene copolymer elastomers, or ethylene/propylene/diene terpolymer elastomers), olefin block copolymers, polyamide elastomers, ethylene vinyl acetate elastomers, and polyester elastomers. An ABA block copolymer elastomer generally is one where the A blocks are polystyrenic, and the B blocks are prepared from conjugated dienes (e.g., lower alkylene dienes). The A block is generally formed predominantly of substituted (e.g., alkylated) or unsubstituted styrenic moieties (e.g., polystyrene, poly(alphamethylstyrene), or poly(t-butylstyrene)), having an average molecular weight from about 4.000 to 50,000 grams per mole. The B block(s) is generally formed predominantly of conjugated dienes (e.g., isoprene, 1,3-butadiene, or ethylene-butylene monomers), which may be substituted or unsubstituted, and has an average molecular weight from about 5,000 to 500,000 grams per mole. The A and B blocks may be configured, for example, in linear, radial, or star configurations. An ABA block copolymer may contain multiple A and/or B blocks, which blocks may be made from the same or different monomers. A typical block copolymer is a linear ABA block copolymer, where the A blocks may be the same or different, or a block copolymer having more than three blocks, predominantly terminating with A blocks. Multi-block copolymers may contain, for example, a certain proportion of AB diblock copolymer, which tends to form a tackier elastomeric film segment. Other elastic polymers can be blended with block copolymer elastomers, and various elastic polymers may be blended to have varying degrees of elastic properties. Blends of these elastomers with each other or with modifying non-elastomers are also contemplated. Many types of thermoplastic elastomers are commercially available, including those from BASF, Florham Park. N.J., under the trade designation “STYROFLEX”, from Kraton Polymers, Houston, Tex., under the trade designation “KRATON”, from Dow Chemical, Midland, Mich., under the trade designation “PELLETHANE”, “INFUSE”, VERSIFY”, or “NORDE.”, from DSM, Heerlen, Netherlands, under the trade designation “ARNITEL”, from E. I. duPont de Nemours and Company, Wilmington, Del., under the trade designation “HYTREL”, from ExxonMobil, Irving. Tex. under the trade designation “VISTAMAXX”, and more.

In some embodiments, the elastic layer is a multilayer film. In some embodiments, the elastic layer comprises two skin layers and an elastomeric core layer sandwiched therebetween. The multilayer film is relatively inelastic prior to activation. However, the film can be rendered elastic by stretching the multilayer film past the elastic deformation limit of the skin layers and recovering the skin layers with the elastomeric core layer to produce a multilayer film that is elastic in the direction of stretch. Due to the deformation of the skin layers during activation, the multilayer film exhibits a microtextured surface upon recovery. Microtexture refers to the structure of the skin layers in the area of activation. More particularly, the skin layers contain peak and valley irregularities or folds, the details of which typically cannot be seen without magnification.

The skin layers can be formed of any semi-crystalline or amorphous polymer that is less elastic than the elastomeric core layer and will undergo permanent deformation at the desired percent stretch of the multilayer film. Therefore, slightly elastomeric compounds, such as some olefinic elastomers, e.g. ethylene-propylene elastomers or ethylene-propylene-diene terpolymer elastomers or ethylenic copolymers, e.g., ethylene vinyl acetate, can be used as skin layers, either alone or in blends. However, the skin layer is generally a polyolefin such as polyethylene, polypropylene, polybutylene or a polyethylene-polypropylene copolymer, but may also be wholly or partly polyamide such as nylon, polyester such as polyethylene terephthalate, polyvinylidene fluoride, polyacrylate such as poly(methyl methacrylate) (generally in blends), and blends thereof. The skin and core layers may be in substantially continuous contact so as to minimize the possibility of delamination of the skin layers from the core layer, but this is not a requirement. The multilayer films can conveniently be prepared by coextrusion of the elastomeric core layer and skin layers although other methods of preparing the multilayer film are possible.

In some embodiments of the multilayer elastic film useful for practicing the present disclosure, the core layer of the multilayer film is a styrenic block copolymer and the skin layers of the multilayer film are each a polyolefin. In other embodiments, the core layer of the multilayer film is a styrene-isoprene-styrene (SIS) and polystyrene blend and the skin layers of the multilayer film are each a polypropylene and polyethylene blend. In yet other embodiments, the core layer of the multilayer film is a SIS and polystyrene blend and the skin layers of the multilayer film are each polypropylene.

Other layers may be added between the elastomeric core layer and the skin layers, such as tie layers, to improve the bonding of the skin and core layers. Tie layers can be formed of, or compounded with, for example, maleic anhydride modified elastomers, ethyl vinyl acetates and olefins, polyacrylic imides, butyl acrylates, peroxides such as peroxypolymers (e.g., peroxyolefins) silanes (epoxysilanes), reactive polystyrenes, chlorinated polyethylene, acrylic acid modified polyolefins, and ethyl vinyl acetates with acetate and anhydride functional groups, which can also be used in blends or as compatibilizers or adhesion-promoting additives in one or more of the skin or core layers.

The core:skin thickness ratio of the multilayer films is typically selected to allow for an essentially homogeneous activation of the multilayer film. The core:skin thickness ratio is defined as the ratio of the thickness of the elastomeric core layer over the sum of the thicknesses of the two skin layers. Additionally, the core:skin thickness ratio of the multilayer film can be selected so that when the skin layers are stretched beyond their elastic deformation limit and relaxed with the elastomeric core layer, the skin layers form a microtextured surface. The desired core:skin ratio will depend upon several factors, including the composition of the film. In some embodiments of the multilayer elastic film useful for practicing the present disclosure, the core:skin ratio of the multilayer film is at least 2:1. In other embodiments, the core:skin ratio of the multilayer film is at least 3:1.

The skin layers of the multilayer elastic films may be the same composition or different. Similarly, the skin layers may be the same thickness or different. In some embodiments, the skin layers have the same composition and thickness.

Examples of multilayer films useful for practicing the present disclosure are described in U.S. Pat. No. 5,462,708 (Swenson, et al.); U.S. Pat. No. 5,344,691 (Hanschen, et al.); U.S. Pat. No. 5,501,679 (Krueger, et al.), and U.S. Pat. No. 9,469,091 (Henke et al.). Suitable commercially available multilayer elastic films useful for practicing the present disclosure include M-235 available from 3M Company in St. Paul, Minn., USA.

Viscosity reducing polymers and plasticizers can also be blended with the elastomers useful for making the elastic fibers, strands, and films. Viscosity reducing polymers include low molecular weight polyethylene and polypropylene polymers and copolymers and tackifying resins. Tackifiers can also be used to increase the adhesiveness of an elastomeric core layer to a skin layer in the multilayer films described above. Examples of tackifiers include aliphatic or aromatic hydrocarbon liquid tackifiers, polyterpene resin tackifiers, and hydrogenated tackifying resins.

Additives such as dyes, pigments, antioxidants, antistatic agents, bonding aids, fillers, antiblocking agents, slip agents, heat stabilizers, photostabilizers, foaming agents, glass bubbles, reinforcing fiber, starch and metal salts for degradability, microfibers, and extenders (e.g., mineral oil extenders) can also be used in the elastic layer or at least a portion thereof.

When the elastic layer is a multilayer film having an elastic core and two opposing less elastic skin layers, the process for making the composite elastic material of the present disclosure can include stretching the elastic layer in a direction perpendicular to the first direction to plastically deform the skin layers and then allowing the elastic layer to relax. This process is carried out before stretching the elastic layer in the first direction and can be referred to as “activation”. This activation can advantageously reduce the necking of the multilayer film during stretching in the first direction when contrasted with an nonactivated multilayer film. Reduced necking typically results in greater recovery of the multilayer film after stretching in the first direction and hence more efficient use of the elastic material. Reduced necking also reduces width variability of the multilayer film during processing, thus reducing film and laminate waste and improving process handling capabilities. In addition, the activated multilayer film is relatively inelastic in the first direction before being stretched in the first direction and would therefore be less subject to premature stretching on a manufacturing line.

A versatile stretching method that allows for monoaxial, sequential biaxial, and simultaneous biaxial stretching of the multilayer film employs a flat film tenter apparatus, described above. Flat film tenter stretching apparatuses are commercially available, for example, from Brückner Maschinenbau GmbH, Siegsdorf, Germany. Cross-direction stretching of the multilayer elastic film can also be using diverging disks, diverging rails, and incremental stretching devices, for example. Incremental stretching of the laminate can be carried out in any one of a variety of ways including ring-rolling, structural elastic film processing (SELFing), which may be differential or profiled, in which not all material is strained in the direction of stretching, and other means of incrementally stretching webs as known in the art. An example of a suitable incremental activation process is the ring-rolling process, described in U.S. Pat. No. 5,366,782 (Curro). Specifically, a ring-rolling apparatus includes opposing rolls having intermeshing teeth that incrementally stretch and can plastically deform the fibrous web (or a portion thereof), rendering the fibrous web stretchable in the ring-rolled regions. These opposing rolls can be considered to be corrugated rolls that provide the intermeshing surfaces through which the multilayer elastic film is passed. In another example of a suitable incremental stretching device, the intermeshing surfaces are intermeshing discs, which may be mounted, for example, at spaced apart locations along a shaft as shown, for example, in U.S. Pat. No. 4,087,226 (Mercer). The intermeshing surfaces can also include rotating discs that intermesh with a stationary, grooved shoe.

The degree of stretch imparted to the film can be represented by the stretch ratio. Stretch ratio in the context of cross-direction activation is defined as the width of the stretched film to the width of the unstretched film. The typical stretch ratio is more than required to stretch the skin layers beyond the elastic deformation limit but less than that required to permanently deform the elastic core layer beyond a small permanent set. In some embodiments, the stretch ratio of the multilayer film ranges from 2:1 to 5:1. Cross-direction activation of the multilayer film can be performed in-line with the apparatus used to make the composite elastic material. Alternatively, cross-direction activation can be performed off-line and the activated multilayer film supplied in roll form as elastic web 4 in FIG. 1, for example.

The structured film useful for the composite elastic material and process of making a composite elastic material according to the present disclosure may be made from a variety of suitable materials. In some embodiments, the structured film is a thermoplastic film. Suitable thermoplastic materials include polyolefin homopolymers such as polyethylene and polypropylene, copolymers of ethylene, propylene and/or butylene; copolymers containing ethylene such as ethylene vinyl acetate and ethylene acrylic acid; polyesters such as poly(ethylene terephthalate), polyethylene butyrate and polyethylene naphthalate; polyamides such as poly(hexamethylene adipamide); polyurethanes; polycarbonates; poly(vinyl alcohol); ketones such as polyetheretherketone; polyphenylene sulfide; and mixtures thereof. In some embodiments, the thermoplastic is a polyolefin (e.g., polyethylene, polypropylene, polybutylene, ethylene copolymers, propylene copolymers, butylene copolymers, and copolymers and blends of these materials). For any of the embodiments in which the thermoplastic backing includes polypropylene, the polypropylene may include alpha and/or beta phase polypropylene.

In some embodiments, the structured film can be made from a multilayer or multi-component melt stream of thermoplastic materials. This can result in surface structures formed at least partially from a different thermoplastic material than the one predominately forming the backing. Various configurations of upstanding posts made from a multilayer melt stream are shown in U.S. Pat. No. 6,106,922 (Cejka et al.), for example. A multilayer or multi-component melt stream can be formed by any conventional method. A multilayer melt stream can be formed by a multilayer feedblock, such as that shown in U.S. Pat. No. 4,839,131 (Cloeren). A multicomponent melt stream having domains or regions with different components could also be used. Useful multicomponent melt streams could be formed by use of inclusion co-extrusion die or other known methods (e.g., that shown in U.S. Pat. No. 6,767,492 (Norquist et al.).

Structured films useful for practicing the present disclosure typically have a backing and upstanding male fastening elements that are integral (that is, generally formed at the same time as a unit, unitary). The term “upstanding” refers to male fastening elements that protrude from a backing that stand perpendicular to the backing and male fastening elements that are at an angle to the backing other than 90 degrees. Upstanding posts on a backing can be made, for example, by feeding a thermoplastic material onto a continuously moving mold surface with cavities having the inverse shape of the male fastening elements or a precursor of the male fastening elements. The thermoplastic material can be passed between a nip formed by two rolls or a nip between a die face and roll surface, with at least one of the rolls having the cavities. Pressure provided by the nip forces the resin into the cavities. In some embodiments, a vacuum can be used to evacuate the cavities for easier filling of the cavities. The nip has a large enough gap such that a coherent thermoplastic backing is formed over the cavities. The mold surface and cavities can optionally be air or water cooled before stripping the integrally formed backing and upstanding posts from the mold surface such as by a stripper roll.

Mold surfaces suitable for forming structured surfaces can be made, for example, by forming (e.g., by computer numerical control with drilling, photo etching, using galvanic printed sleeves, laser drilling, electron beam drilling, metal punching, direct machining, or lost wax processing) a series of cavities having the inverse shape of the male fastening elements or precursor of the male fastening elements into the cylindrical face of a metal mold or sleeve. Suitable tool rolls include such as those formed from a series of plates defining a plurality of cavities about its periphery including those described, for example, in U.S. Pat. No. 4,775,310 (Fischer). Cavities may be formed in the plates by drilling or photoresist technology, for example. Other suitable tool rolls may include wire-wrapped rolls, which are disclosed along with their method of manufacturing, for example, in U.S. Pat. No. 6,190,594 (Gorman et al.). Another example of a method for forming a thermoplastic backing with male fastening elements includes using a flexible mold belt defining an array of cavities as described in U.S. Pat. No. 7,214,334 (Jens et al.). Yet other useful methods for forming a thermoplastic backing with male fastening elements can be found in U.S. Pat. No. 6,287,665 (Hammer), U.S. Pat. No. 7,198,743 (Tuma), and U.S. Pat. No. 6,627,133 (Tuma).

In any of the mold surfaces mentioned above, the cavities and the resultant male fastening elements may have a variety of cross-sectional shapes. For example, the cross-sectional shape of the cavity and surface structure may be a polygon (e.g., square, rectangle, rhombus, hexagon, pentagon, or dodecagon), which may be a regular polygon or not, or the cross-sectional shape of the cavity and surface structure may be curved (e.g., round or elliptical). The surface structure may taper from its base to its distal tip, for example, for easier removal from the cavity, but this is not a requirement.

With reference to any of the mold surfaces described above, the cavity may have the inverse shape of a post having a loop-engaging head (e.g., a male fastening element) or may have the inverse shape of an upstanding post without loop-engaging heads that can be formed into loop-engaging heads, if desired. If upstanding posts formed upon exiting the cavities do not have loop-engaging heads, loop-engaging heads could be subsequently formed by a capping method as described in U.S. Pat. No. 5,077,870 (Melbye et al.). Typically, the capping method includes deforming the tip portions of the upstanding posts using heat and/or pressure. The heat and pressure, if both are used, could be applied sequentially or simultaneously. The formation of male fastening elements can also include a step in which the shape of the cap is changed, for example, as described in U.S. Pat. No. 6,132,660 (Kampfer).

For any of the embodiments described above in which the surface structures are upstanding posts with loop-engaging overhangs, the term “loop-engaging” relates to the ability of a male fastening element to be mechanically attached to a loop material. Generally, male fastening elements with loop-engaging heads have a head shape that is different from the shape of the post. For example, the male fastening element may be in the shape of a mushroom (e.g., with a circular or oval head enlarged with respect to the stem), a hook, a palm-tree, a nail, a T. or a J. In some embodiments, useful loop engaging overhangs extend in multiple (i.e., at least two) directions, in some embodiments, at least two orthogonal directions. For example, the upstanding post may be in the shape of a mushroom, a nail, a palm tree, or a T. In some embodiments, the upstanding posts are provided with a mushroom head (e.g., with an oval or round cap distal from the thermoplastic backing). The loop-engageability of male fastening elements may be determined and defined by using standard woven, nonwoven, or knit materials. A region of male fastening elements with loop-engaging heads generally will provide, in combination with a loop material, at least one of a higher peel strength, higher dynamic shear strength, or higher dynamic friction than a region of posts without loop-engaging heads. Male fastening elements that have “loop-engaging overhangs” or “loop-engaging heads” do not include ribs that are precursors to fastening elements (e.g., elongate ribs that are profile extruded and subsequently cut to form male fastening elements upon stretching in the direction of the ribs). Such ribs would not be able to engage loops before they are cut and stretched. Such ribs would also not be considered upstanding posts. Typically, male fastening elements that have loop-engaging heads have a maximum width dimension (in either dimension normal to the height) of up to about 1 (in some embodiments, 0.9, 0.8, 0.7, 0.6, 0.5, or 0.45) millimeter. In some embodiments, the male fastening elements have a maximum height (above the backing) of up to 3 mm, 1.5 mm, 1 mm, or 0.5 mm and, in some embodiments a minimum height of at least 0.03 mm, 0.05 mm, 0.1 mm, or 0.2 mm. In some embodiments, the upstanding posts have aspect ratio (that is, a ratio of height to width at the widest point) of at least about 0.25:1, 1:1, 2:1, 3:1, or 4:1.

In the structured film layer useful for practicing the present disclosure, male fastening elements are typically spaced apart on a backing. The term “spaced-apart” refers to male fastening elements that are formed to have a distance between them. The bases of “spaced-apart” surface structures, where they are attached to the backing, do not touch each other when the backing is in an unbent configuration. The backing in these embodiments may be considered to be an unstructured film region or as an aggregate of unstructured film regions. Spaced-apart male fastening elements may have a density of at least 10 per square centimeter (cm²) (63 per square inch in²). For example, the density of the spaced-apart surface structures may be at least 100/cm² (635/in²), 248/cm² (1600/in²), 394/cm² (2500/in²), or 550/cm² (3500/in²). In some embodiments, the density of the spaced-apart surface structures may be up to 1575/cm² (10000/in²), up to about 1182/cm² (7500/in²), or up to about 787/cm² (5000/in²). Densities in a range from 10/cm² (63/in²) to 1575/cm² (10000/in²) or 100/cm² (635/in²) to 1182/cm² (7500/in²) may be useful, for example. The spacing of the spaced-apart male fastening elements need not be uniform.

In some embodiments of the structured film layer useful for practicing the present disclosure, the structured film layer has been stretched, for example, before being bonded to the elastic layer. Stretching can be useful, for example, for decreasing the thickness of the structured film layer and providing thinner and more flexible composite elastic material. Stretching the structured film can be carried out using a variety of methods. Stretching in the machine direction of a continuous web of indefinite length, can be performed by propelling the web over rolls of increasing speed, with the downweb roll speed faster than the upweb roll speed. Stretching in a cross-machine direction can be carried out on a continuous web using, for example, diverging rails, diverging disks, a series of bowed rollers, a crown surface, or a tenter apparatus as described above in connection with stretching the elastic layer. Monoaxial and biaxial stretching can also be accomplished, for example, by the methods and apparatus disclosed in U.S. Pat. No. 7,897,078 (Petersen et al.) and the references cited therein. Useful draw ratios can include at least 1.25, 1.5, 2.0, 2.25, 2.5, 2.75, or 3, and draw ratios of up to 5, 7.5, or 10 may be useful, depending on material selection and the temperature of the thermoplastic backing when it is stretched.

Heating the structured film may be useful, for example, before or during stretching. This may allow the structured film to be more flexible for stretching. Heating can be provided, for example, by IR irradiation, hot air treatment or by performing the stretching in a heat chamber. In some embodiments, heating is only applied to the second surface of the film (i.e., the surface opposite the first surface having upstanding male fastening elements) to minimize any damage to the surface structures that may result from heating. In some embodiments in which the structured film comprises polypropylene, stretching is carried out in a temperature range from 50° C. to 130° C., 50° C. to 110° C. 80° C. to 110° C., 85° C. to 100° C., or 90° C. to 95° C.

In some embodiments, including any of those embodiments in which the structured film layer is stretched, the density of the upstanding male fastening elements is lower than before stretching. In some of these embodiments, the upstanding male fastening elements have a density of at least 2 per square centimeter (cm²) (13 per square inch in²). For example, in some of these embodiments, the density of the male fastening elements may be at least 62/cm² (400/in²), 124/cm² (800/in²) 248/cm² (1600/in²), or 394/cm² (2500/in²) and may be up to about 1182/cm² (7500/in²) or up to about 787/cm² (5000/in²). Useful densities of male fastening elements in a stretched structured film layer include those in a range from 2/cm² (13/in²) to 1182/cm² (7500/in²) or 124/cm² (800/in²) to 787/cm² (5000/in²), for example. Again, the spacing of the surface structures need not be uniform.

In some embodiments in which the structured film layer is stretched, it has stretch-induced molecular orientation. Stretch-induced molecular orientation in the structured film can be determined by standard spectrographic analysis of the birefringent properties of the film. Birefringence refers to a property of a material having different effective indexes of refraction in different directions. In the present application, birefringence is evaluated with a retardance imaging system available from Lot-Oriel GmbH & Co., Darmstadt, Germany, under the trade designation “LC-POLSCOPE” on a microscope available from Leica Microsystems GmbH, Wetzlar, Germany, under the trade designation “DMRXE” and a digital CCD color camera available from OImaging, Surrey, BC, Canada, under the trade designation “RETIGA EXi FAST 1394”. The microscope is equipped with a 546.5 nm interference filter obtained from Cambridge Research & Instrumentation, Inc., Hopkinton, Mass., and 10×/0.25 objective.

The male fastening elements may be provided in a variety of patterns. For example, there may be groups of male fastening elements clustered together, with separation between the clusters. The male fastening elements may also be provided in square arrays or staggered arrays, for example.

In the composite elastic material according to the present disclosure, the structured film layer is gathered such that the upstanding male fastening elements point in multiple directions. In the process for making the composite elastic material of the present disclosure, the elastic layer is allowed to relax and the structured film layer allowed to gather. The spacing between gathers can be indicative of how well a composite elastic material according to the present disclosure functions as an elastic, that is, how well it can extend and recover. In some embodiments, the spacing between gathers in the structured film layer is up to five, four, three, or two millimeters. Structured film layers that are too stiff to gather well, thereby hindering the ability of the composite elastic material to stretch and relax, may have a spacing between gathers of greater than a centimeter, greater than two centimeters, or more. The spacing between gathers can be conveniently measured as the distance between midpoints of adjacent gathers. Alternatively, the spacing between gathers can conveniently be evaluated as a number of gathers per centimeter. For structured films that do not gather well, the number of gathers per centimeter may be less than 1 when the composite elastic material is fully relaxed. For structured films that gather well, the number of gathers per centimeter may be greater than 1, 1.5, or 2 when the composite elastic material is fully relaxed. More gathers in the structured film layer can also provide an improved look and feel for the composite elastic material.

Various features of the structured film may be useful to facilitate gathering when tension on the composite elastic material is released. These include the selection of materials, the thickness of the structured film (typically excluding the male fastening elements), the presence of pores in the film, and the presence of discontinuities in the structured film backing.

Material selection can influence how well a structured film layer gathers in the composite elastic material of the present disclosure and/or the process for making the composite elastic material. In some embodiments, the structured film useful in the composite elastic material and process according to the present disclosure comprises polypropylene. Semi-crystalline polyolefins can have mom than one kind of crystal structure. For example, isotactic polypropylene is known to crystallize into at least three different forms: alpha (monoclinic), beta (pseudohexangonal), and gamma (triclinic) forms. In melt-crystallized material the predominant form is the alpha or monoclinic form. The beta form generally occurs at levels of only a few percent unless certain heterogeneous nuclei are present or the crystallization has occurred in a temperature gradient or in the presence of shearing forces. These certain heterogeneous nuclei are typically known as beta-nucleating agents, which act as foreign bodies in a crystallizable polymer melt. When the polymer cools below its crystallization temperature (e.g., a temperature in a range from 60° C. to 120° C. or 90° C. to 120° C.), the loose coiled polymer chains orient themselves around the beta-nucleating agent to form beta-phase regions. The beta form of polypropylene is a meta-stable form, which can be converted to the more stable alpha form by thermal treatment and/or applying stress. In some embodiments, the structured film comprises a beta-nucleating agent. Micropores can be formed in various amounts when the beta-form of polypropylene is stretched under certain conditions; see, e.g., Chu et al., “Microvoid formation process during the plastic deformation of β-form polypropylene”, Polymer, Vol. 35, No. 16, pp. 3442-3448, 1994, and Chu et al., “Crystal transformation and micropore formation during uniaxial drawing of f-form polypropylene film”, Polymer, Vol. 36. No. 13. pp. 2523-2530, 1995. Pore sizes achieved from this method can range from about 0.05 micrometer to about 1 micrometer, in some embodiments, about 0.1 micrometer to about 0.5 micrometer. In some embodiments, the structured film layer includes a beta-nucleating agent, and/or at least a portion of the structured film layer includes beta-spherulites. In some embodiments, at least a portion of the structured film layer is microporous.

Generally, when the structured film comprises a beta-nucleating agent, the structured film comprises polypropylene. It should be understood that a structured film comprising polypropylene may comprise a polypropylene homopolymer or a copolymer containing propylene repeating units. The copolymer may be a copolymer of propylene and at least one other olefin (e.g., ethylene or an alpha-olefin having from 4 to 12 or 4 to 8 carbon atoms). Copolymers of ethylene, propylene and/or butylene may be useful. In some embodiments, the copolymer contains up to 90, 80, 70, 60, or 50 percent by weight of polypropylene. In some embodiments, the copolymer contains up to 50, 40, 30, 20, or 10 percent by weight of at least one of polyethylene or an alpha-olefin. The structured film may also comprise a blend of thermoplastic polymers that includes polypropylene. Suitable thermoplastic polymers include crystallizable polymers that are typically melt processable under conventional processing conditions. That is, on heating, they will typically soften and/or melt to permit processing in conventional equipment, such as an extruder, to form a sheet. Crystallizable polymers, upon cooling their melt under controlled conditions, spontaneously form geometrically regular and ordered chemical structures. Examples of suitable crystallizable thermoplastic polymers include addition polymers, such as polyolefins. Useful polyolefins include polymers of ethylene (e.g., high density, polyethylene, low density polyethylene, or linear low density polyethylene), an alpha-olefin (e.g. 1-butene, 1-hexene, or 1-octene), styrene, and copolymers of two or more such olefins. The semi-crystalline polyolefin may comprise mixtures of stereo-isomers of such polymers, e.g., mixtures of isotactic polypropylene and atactic polypropylene or of isotactic polystyrene and atactic polystyrene. In some embodiments, the semi-crystalline polyolefin blend contains up to 90, 80, 70, 60, or 50 percent by weight of polypropylene. In some embodiments, the blend contains up to 50, 40, 30, 20, or 10 percent by weight of at least one of polyethylene or an alpha-olefin.

In embodiments of the composite elastic material and process according to the present disclosure in which the structured film comprises a beta-nucleating agent, the beta-nucleating agent may be any inorganic or organic nucleating agent that can produce beta-spherulites in a melt-formed sheet comprising polyolefin. Useful beta-nucleating agents include gamma quinacridone, an aluminum salt of quinizarin sulphonic acid, dihydroquinoacridin-dione and quinacridin-tetrone, triphenenol ditriazine, calcium silicate, dicarboxylic acids (e.g., suberic, pimelic, ortho-phthalic, isophthalic, and terephthalic acid), sodium salts of these dicarboxylic acids, salts of these dicarboxylic acids and the metals of Group IIA of the periodic table (e.g., calcium, magnesium, or barium), delta-quinacridone, diamides of adipic or suberic acids, different types of indigosol and cibantine organic pigments, quinacridone quinone, N′,N′-dicyclohexil-2,6-naphthalene dicarboxamide (available, for example, under the trade designation “NJ-Star NU-100” from New Japan Chemical Co. Ltd.), anthraquinone red, and bis-azo yellow pigments. The properties of the extruded film are dependent on the selection of the beta nucleating agent and the concentration of the beta-nucleating agent. In some embodiments, the beta-nucleating agent is selected from the group consisting of gamma-quinacridone, a calcium salt of suberic acid, a calcium salt of pimelic acid and calcium and barium salts of polycarboxylic acids. In some embodiments, the beta-nucleating agent is quinacridone colorant Permanent Red E3B, which is also referred to as Q-dye. In some embodiments, the beta-nucleating agent is formed by mixing an organic dicarboxylic acid (e.g., pimelic acid, azelaic acid, o-phthalic acid, terephthalic acid, and isophthalic acid) and an oxide, hydroxide, or acid salt of a Group 11 metal (e.g., magnesium, calcium, strontium, and barium). So-called two component initiators include calcium carbonate combined with any of the organic dicarboxylic acids listed above and calcium stearate combined with pimelic acid. In some embodiments, the beta-nucleating agent is aromatic tri-carboxamide as described in U.S. Pat. No. 7,423,088 (Milder et al.).

A convenient way of incorporating beta-nucleating agents into a semi-crystalline polyolefin useful for making a structured film disclosed herein is through the use of a concentrate. A concentrate is typically a highly loaded, pelletized polypropylene resin containing a higher concentration of nucleating agent than is desired in the final microporous film. The nucleating agent is present in the concentrate in a range of 0.01% to 2.0% by weight (100 to 20,000 ppm), in some embodiments in a range of 0.02% to 1% by weight (200 to 10,000 ppm). Typical concentrates are blended with non-nucleated polyolefin in the range of 0.5% to 50% (in some embodiments, in the range of 1% to 10%) by weight of the total polyolefin content of the microporous film. The concentration range of the beta-nucleating agent in the final microporous film may be 0.0001% to 1% by weight (1 ppm to 10,000 ppm), in some embodiments, 0.0002% to 0.1% by weight (2 ppm to 1000 ppm). A concentrate can also contain other additives such as stabilizers, pigments, and processing agents.

The level of beta-spherulites in the structured film can be determined, for example, using X-ray crystallography and Differential Scanning Calorimetry (DSC). By DSC, melting points and heats of fusion of both the alpha phase and the beta phase can be determined in a structured film useful for practicing the present disclosure. For semi-crystalline polypropylene, the melting point of the beta phase is lower than the melting point of the alpha phase (e.g., by about 10 to 15 degrees Celsius). The ratio of the heat of fusion of the beta phase to the total heat of fusion provides a percentage of the beta-spherulites in a sample. The level of beta-spherulites can be at least 10, 20, 25, 30, 40, or 50 percent, based on the total amount of alpha and beta phase crystals in the film. These levels of beta-spherulites may be found in the film before it is stretched.

As described above, when the structured film includes a beta-nucleating agent, stretching the film provides micropores in at least a portion of the film. Without wanting to be bound by theory, it is believed that when the film is stretched in at least one direction, for example, the semi-crystalline polypropylene converts from the beta-crystalline structure to the alpha-crystalline structure in the film, and micropores are formed in the film. Upstanding male fastening elements are typically affected differently from the rest of the film. For example, male fastening elements on a backing are typically not affected by the stretching or are affected to a much lesser extent than the backing and therefore retain beta-crystalline structure and generally have lower levels of microporosity than the backing. The resulting stretched films can have several unique properties. For example, the micropores formed in the film along with stress-whitening can provide an opaque, white film with transparent upstanding male fastening elements.

In some embodiments, stretching a structured film layer including a beta-nucleating agent is carried out at temperature range from 50° C. to 110° C., 50° C. to 90° C., or 50° C. to 80° C. In some embodiments, stretching at lower temperatures may be possible, for example, in a range from 25° C. to 50° C. Structured polypropylene films containing a beta-nucleating agent can be stretched at a temperature of up to 70° C. (e.g., in a range from 50° C. to 70° C. or 60° C. to 70° C.) and still successfully achieve microporosity.

As shown in the Examples, below. Example 2, in which the structured film layer was microporous and included a beta-nucleating agent, and/or in which at least a portion of the structured film layer included beta-spherulites had more gathers per centimeter than structured films that were not microporous.

When micropores are formed in the backing of the stretched structured film layer disclosed herein, the density of the film decreases. The resulting low-density stretched structured film layer feels softer to the touch than films having comparable thicknesses but higher densities. The density of the film can be measured using conventional methods, for example, using helium in a pycnometer. The softness of the film can be measured, for example, using Gurley stiffness.

The microporosity that is beneficial for allowing the structured film layer to gather when tension is released from the composite elastic material may be formed by other ways. In some embodiments, the structured film layer useful for practicing the present disclosure in any of its embodiments is formed using a thermally induced phase separation (TIPS) method. This method of making a film typically includes melt blending a crystallizable polymer and a diluent to form a melt mixture. The melt mixture is then formed into a film and cooled to a temperature at which the polymer crystallizes, and phase separation occurs between the polymer and diluent, forming voids. In this manner a film is formed that comprises an aggregate of crystallized polymer in the diluent compound. The voided film has some degree of opacity. In some embodiments, following formation of the crystallized polymer, the porosity of the material is increased by at least one of stretching the film in at least one direction or removing at least some of the diluent. This step results in separation of adjacent particles of polymer from one another to provide a network of interconnected micropores. This step also permanently attenuates the polymer to form fibrils, imparting strength and porosity to the film. The diluent can be removed from the material either before or after stretching. In some embodiments, the diluent is not removed. Pore sizes achieved from this method can range from about 0.2 micron to about 5 microns.

When the structured film useful for practicing the present disclosure is made from a TIPS process, the structured film can comprise any of the semi-crystalline polyolefins described above in connection with films made by beta-nucleation. In addition, other crystallizable polymers that may be useful alone or in combination include high and low density polyethylene, poly(vinylidine fluoride), poly(methyl pentene)(e.g., poly(4-methylpentene), poly(lactic acid), poly(hydroxybutyrate), poly(ethylene-chlorotrifluoroethylene), poly(vinyl fluoride), polyvinyl chloride, poly(ethylene terephthalate), poly(butylene terephthalate), ethylene-vinyl alcohol copolymers, ethylene-vinyl acetate copolymers, polybuyltene, polyurethanes, and polyamides (e.g., nylon-6 or nylon-66). Useful diluents for providing the microporous film include mineral oil, mineral spirits, dioctylphthalate, liquid paraffins, paraffin wax, glycerin, petroleum jelly, polyethylene oxide, polypropylene oxide, polytetramethylene oxide, soft carbowax, and combinations thereof. The quantity of diluent is typically in a range from about 20 parts to 70 parts, 30 parts to 70 parts, or 50 parts to 65 parts by weight, based upon the total weight of the polymer and diluent.

In some embodiments, the structured film layer useful for practicing the present disclosure in any of its embodiments is formed using particulate cavitating agents. Such cavitating agents are incompatible or immiscible with the polymeric matrix material and form a dispersed phase within the polymeric core matrix material before extrusion and orientation of the film. When such a polymer substrate is subjected to uniaxial or biaxial stretching, a void or cavity forms around the distributed, dispersed-phase moieties, providing a film having a matrix filled with numerous cavities that provide an opaque appearance due to the scattering of light within the matrix and cavities. The microporous film can comprise any of the polymers described above in connection with TIPS films. The particulate cavitating agents may be inorganic or organic. Organic cavitating agents generally have a melting point that is higher than the melting point of the film matrix material. Useful organic cavitating agents include polyesters (e.g., polybutylene terephthalate or nylon such as nylon-6), polycarbonate, acrylic resins, and ethylene norbornene copolymers. Useful inorganic cavitating agents include talc, calcium carbonate, titanium dioxide, barium sulfate, glass heads, glass bubbles (that is, hollow glass spheres), ceramic beads, ceramic bubbles, and metal particulates. The particle size of cavitating agents is such that at least a majority by weight of the particles comprise an overall mean particle diameter, for example, of from about 0.1 micron to about 5 microns, in some embodiments, from about 0.2 micron to about 2 microns. (The term “overall” refers to size in three dimensions; the term “mean” is the average.) The cavitating agent may be present in the polymer matrix in an amount of from about 2 weight percent to about 40 weight percent, about 4 weight percent to about 30 weight percent, or about 4 weight percent to about 20 weight percent, based upon the total weight of the polymer and cavitating agent.

Porosity in a structured film layer may also be introduced using physical or chemical blowing agents. Physical or chemical blowing agents are useful in the structured film layer form distinct gas phases. A blowing agent may be any material that is capable of forming a vapor at the temperature and pressure at which an extrudate exits the die during film formation. A blowing agent may be a physical blowing agent. A physical blowing agent may be introduced (e.g., injected) into the thermoplastic material as a gas or supercritical fluid. Flammable blowing agents such as pentane, butane and other organic materials may be used, but non-flammable, non-toxic, non-ozone depleting blowing agents such as carbon dioxide, nitrogen, water, SF₆, nitrous oxide, helium, noble gases (e.g., argon, xenon), air (nitrogen and oxygen blend), and blends of these materials may be easier to use and provide fewer environmental and safety concerns. Other suitable physical blowing agents include hydrofluorocarbons (HFC), hydrochlorofluorocarbons (HCFC), and fully- or partially fluorinated ethers.

A chemical blowing agent may be added to the thermoplastic resin at a temperature below that of the activation temperature of the blowing agent and is typically added to the thermoplastic resin feed at room temperature before introduction to the extruder. The blowing agent is then mixed to distribute it throughout the polymer in nonactivated form, above the melt temperature of the thermoplastic resin but below the activation temperature of the chemical blowing agent. Once dispersed, the chemical blowing agent may be activated by heating the mixture to a temperature above the activation temperature of the agent. Activation of the blowing agent liberates gas (e.g., N₂, CO₂, or H₂O) either through decomposition (e.g., exothermic chemical blowing agents such as azodicarbonamide) or reaction (e.g., endothermic chemical blowing agents such as sodium bicarbonate-citric acid mixtures). Examples of suitable chemical blowing agents include synthetic azo-, carbonate-, and hydrazide based molecules, including azodicarbonamide, azodiisobutyronitrile, benzenesulfonhydrazide, 4,4-oxybenzene sulfonyl-semicarbazide, p-toluene sulfonyl semi-carbazide, barium azodicarboxylate, N,N′-dimethyl-N,N′-dinitrosoterephthalamide trihydrazino triazine and 4,4′oxybis (benzenesulfonylhydrazide)). Other chemical blowing agents include endothermic reactive materials such as sodium bicarbonate/citric acid bends that release carbon dioxide. A specific example includes products obtained under the trade designation “SAFOAM” from Reedy Chemical Foam and Specialty Additives, Charlotte. N.C. Useful chemical blowing agents typically activate at a temperature of at least 140° C.

Cell formation can be restrained by the temperature and pressure of the film during formation. When the extrudate exit temperature is at or below 50° C. above the melting point of the thermoplastic resin, the increase in melting point the resin as the blowing agent comes out of the solution causes crystallization of the thermoplastic resin, which in turn arrests the growth and coalescence of the foam cells. The amount of blowing agent incorporated into the foamable thermoplastic phase is generally chosen to yield a foam having a void content of at least 10%, in some embodiments at least 20%, as measured by density reduction; [1—the ratio of the density of the foam to that of the neat polymer]×100.

Also, when the structured film layer includes microporosity that provides opacity in the film, discontinuously bonding the elastic layer and structured film layer using any of the methods described above can collapse the microporous structure in the bond sites. The bond sites may be see-through regions of lower porosity that contrast with the surrounding opaque, microporous region. The term “see-through”refers to either transparent (that is, allowing passage of light and permitting a clear view of objects beyond) or translucent (that is, allowing passage of light and not permitting a clear view of objects beyond). The see-through region may be colored or colorless. It should be understood that a “see-through” region is large enough to be seen by the naked eye. The elastic layer and/or the optional fibrous layer in some embodiments, may have a contrasting color from the structured film layer that may be visible in the bond sites once the microporous structure is collapsed. Contrasting colors in the structured film layer and the elastic layer and/or the optional fibrous layer may be provided by including a dye or a pigment in at least one of the structured film layer, elastic layer, or optional fibrous layer.

The tendency for the structured film layer to gather can also be increased by incorporating elastomers into the structured film layer. In some embodiments, the structured film layer useful for the composite elastic material of the present disclosure and/or the process for making it is made from a blend of any of thermoplastic materials described above for the structured film layer and an elastomer. Examples of useful elastomers include ABA block copolymers (e.g., in which the A blocks are polystyrenic and formed predominantly of substituted (e.g., alkylated) or unsubstituted moieties and the B blocks are formed predominately from conjugated dienes (e.g., isoprene and 1,3-butadiene), which may be hydrogenated), polyurethane elastomers, polyolefin elastomers (e.g., metallocene polyolefin elastomers), olefin block copolymers, polyamide elastomers, ethylene vinyl acetate elastomers, and polyester elastomers. Examples of useful polyolefin elastomers include an ethylene propylene elastomer, an ethylene octene elastomer, an ethylene propylene diene elastomer, an ethylene propylene octene elastomer, polybutadiene, a butadiene copolymer, polybutene, or a combination thereof. Elastomers are available from a variety of commercial sources as described below. Any of these elastomers may be present in a blend with any of the thermoplastics described above in an amount of up to 20, 15, or 10 percent by weight.

The thickness of the structured film also influences its ability to gather when tension is released from the composite elastic material. As shown in the Examples, below, Example 1, which had a thickness of 60 micrometers had almost three times more gathers per centimeter than Example 3, which had a thickness of 95 micrometers. Although the material selection and presence of pores can influence the useful thickness of the structured film layer, in some embodiments the structured film layer useful for practicing the present disclosure, excluding the upstanding male fastening elements, has a thickness in a range from 20 micrometers to 100 micrometers, 20 micrometers to 80 micrometers, or 30 micrometers to 70 micrometers. A structured film layer may be cast at these film thicknesses, the thickness of the backing can be reduced by stretching the structured film using any of the methods described above.

The tendency for the structured film layer to gather can also be influenced by forming lines of weakness or openings in the structured film layer. A line of weakness may be, for example, a series of perforations or interrupted slits that extend through the backing. The series of perforations typically includes connection points where the backing is not cut through, which prevent the backing from being severed by the lines of weakness. The lines of weakness can be made by a variety of useful slitting methods. Lines of weakness can also be formed as partial-depth cut into the first face of the backing (i.e., the same face from which the male fastening elements project). In some embodiments, the partial-depth slits penetrate the thickness of the backing in a range from 40 to 90 percent. The partial-depth slit may penetrate, for example, 80, 85, or 90 percent of the thickness of the web or more, which means the solution to the equation:

(depth of the slit divided by the thickness of the web)×100

is at least 80, 85, or 90 in some embodiments. Partial-depth cuts can be made by slitting, or the tool useful for making the upstanding male fastening elements may include structures that protrude from the tool and make depressions in the surface of the film. Partial-depth cuts provide a structured film layer (excluding the upstanding posts) having variations in thickness. Typically, the lines of weakness extend perpendicular to the direction of stretch. Lines of weakness are typically made without removing material from the structured film layer. Further details about providing lines of weakness (e.g., interrupted slits or partial-depth slits) in a structured film useful as a mechanical fastener can be found in U.S. Pat. No. 9,138,957 (Wood et al.).

Openings in the structured film layer may also influence the tendency for the structured film layer to gather in the composite elastic material or process for making it disclosed herein. Such openings in the structured film may be useful, for example, for improving the flexibility and/or decreasing the stiffness of the structured film layer. For any of the embodiments of the composite elastic material according to the present disclosure or the process of making the composite elastic material according to the present disclosure, the structured film may include openings. The openings in the structured film layer may be in the form of a repeating pattern of geometric shapes such as circles, ovals, or polygons. The polygons may be, for example, hexagons or quadrilaterals such as parallelograms or diamonds. The openings may be formed in the structured film layer by any suitable method, including die punching. In some embodiments in which the structured film includes openings (e.g., diamond- or hexagonal-shaped openings), the elastic layer or other fibrous layers that may be present do not include openings.

In some embodiments, the openings may be formed by slitting the thermoplastic backing of a structured film layer to form multiple strands attached to each other at intact bridging regions in the backing and separating at least some of the multiple strands between at least some of the bridging regions. The bridging regions are regions where the backing is not cut through, and at least a portion of the bridging regions can be considered collinear with the slits. The intact bridging regions of the backing serve to divide the slits into a series of spaced-apart slit portions aligned in the direction of slitting (e.g., the direction perpendicular to the direction of stretch), which can be referred to as interrupted slits. In some embodiments, for at least some adjacent interrupted slits, the spaced-apart slit portions are staggered in a direction transverse to the slitting direction (e.g., the direction of stretch). The interrupted slits may be cut into the backing between some pairs of adjacent rows of stems although this is not a requirement. In some embodiments, curved lines may be used, which can result in crescent shaped openings after spreading. There may be more than one repeating pattern of geometric shaped openings. The openings may be evenly spaced or unevenly spaced as desired. For openings that are evenly spaced, the spacing between the openings may differ by up to 10, 5, 2.5, or 1 percent. Further details about providing openings in a structured film useful as a mechanical fastener can be found in U.S. Pat. No. 9,138,031 (Wood et al.).

While slits or openings in the structured film layer may be useful in some cases to help the structured film layer to gather when tension is released from the composite elastic material, these are not required. As described above shown in the Examples below, reducing film thickness and material selection, including using a beta-nucleating agent to make a microporous film, are useful for allowing gathers to form in the structured film layer. Accordingly, in some embodiments, the structured film layer is not provided with lines of weakness or openings as described in any of their embodiments above. In some embodiments, the structured film layer is continuous (that is, has no slits or openings therethrough) in at least the direction of stretch. Microporous films can still be continuous since they do not have holes forming a straight path through the entire thickness of the backing of the structured film layer.

In some embodiments, including the embodiments illustrated in FIGS. 1, 2, and 2A, the composite elastic material of the present disclosure and/or made by the process described herein includes at least one fibrous layer bonded to the elastic layer. In some embodiments, the fibrous layer is on a surface of the elastic layer opposite to the surface bonded to the structured film layer. In some embodiments, the fibrous layer is positioned between the elastic layer and the structured film layer. Fibrous layers may also be bonded to both surfaces of the elastic layer. Similarly, in the process of the present disclosure, the process can include bonding at least one fibrous web to the elastic layer while the elastic layer is stretched. The fibrous layer may be continuous (i.e., without any through-penetrating holes) or discontinuous (e.g. comprising through-penetrating perforations or pores). One or more fibrous layers can be included in the composite elastic material to provide a laminate with higher strength, softness, and/or function in comparison to the stretch-bonded structured film layer itself.

The fibrous layer may comprise a variety of suitable materials including woven webs, nonwoven webs, textiles, knit materials, and combinations thereof. Examples of nonwoven webs that may be useful for the fibrous layer include spunbond webs, spunlaced webs, airlaid webs, meltblown web, and bonded carded webs. In some embodiments, the fibrous layer comprises multiple layers of nonwoven materials with, for example, at least one layer of a meltblown nonwoven and at least one layer of a spunbonded nonwoven, or any other suitable combination of nonwoven materials. For example, the fibrous layer may be a spunbond-meltbond-spunbond, spunbond-spunbond, or spunbond-spunbond-spunbond multilayer material. Useful fibrous layers may have any suitable basis weight or thickness that is desired for a particular application. The basis weight may range, e.g., from at least about 5, 8, 10, 20, 30, or 40 grams per square meter, up to about 400, 200, or 100 grams per square meter. The fibrous layer may be up to about 5 mm, about 2 mm, or about 1 mm in thickness and/or at least about 0.1, about 0.2, or about 0.5 mm in thickness. The fibrous layer is typically a gatherable material that forms gathers when the elastic layer recovers from stretching.

In some embodiments, at least the portion of the fibrous layer is not extensible. In some embodiments, at least a portion of the fibrous layer has up to a 10 (in some embodiments, up to 9, 8, 7, 6, or 5) percent elongation in the cross-direction. In other embodiments, one or more zones of the fibrous layer may comprise one or more elastically extensible materials extending in at least one direction when a force is applied and returning to approximately their original dimension after the force is removed. In some embodiments, the fibrous layer may be extensible but non-elastic. In other words, the fibrous layer may have an elongation of at least 5, 10, 15, 20, 25, 30, 40, or 50 percent but substantially no recovery from the elongation (e.g., up to 40, 25, 20, 10, or 5 percent recovery). The term “extensible” refers to a material that can be extended or elongated in the direction of an applied stretching force without destroying the structure of the material or material fibers. In some embodiments, an extensible material may be stretched to a length that is at least about 5, 10, 15, 20, 25, or 50 percent greater than its relaxed length without destroying the structure of the material or material fibers.

In some embodiments of the composite elastic material and process of the present disclosure, the fibrous layer comprises surface loops. The loops may be part of a fibrous structure formed by any of several methods such as weaving, knitting, warp knitting, weft insertion knitting, circular knitting, or methods for making nonwoven structures. In some embodiments, the loops are included in a nonwoven web or a knitted web. Examples of loop tapes that may suitable as fibrous layers for the composite fabric are disclosed, for example, in U.S. Pat. No. 5,389,416 (Mody et al.) and U.S. Pat. No. 5,256,231 (Gorman et al.) and EP 0,341,993 (Gorman et al.). As described in U.S. Pat. No. 5,256,231 (Gorman et al.), the fibrous layer in a loop material can comprise arcuate portions projecting in the same direction from spaced anchor portions on a film. In these embodiments, generally the fibrous layer is adjacent the elastic layer on a surface opposite to the structured film layer.

A variety of suitable materials may be useful for the fibers in the fibrous layer useful for practicing some embodiments of the present disclosure. Examples of suitable materials for forming fibers include polyolefin homopolymers and copolymers; copolymers containing ethylene such as ethylene vinyl acetate and ethylene acrylic acid; polyesters such as poly(ethylene terephthalate), polyethylene butyrate and polyethylene naphthalate; polyamides such as poly(hexamethylene adipamide); polyurethanes; polycarbonates; poly(vinyl alcohol); ketones such as polyetheretherketone; polyphenylene sulfide; viscose; and mixtures thereof. In some embodiments, fibers comprise polyolefins (e.g., polyethylene, polypropylene, polybutylene, ethylene copolymers, propylene copolymers, butylene copolymers, and copolymers and blends of these polymers), polyesters, polyamides, or a combination thereof. The fibers may also be multi-component fibers, for example, having a core of one thermoplastic material and a sheath of another thermoplastic material. The sheath may melt at a lower temperature than the core, providing partial, random bonding between the fibers when the mat of fibers is exposed to a temperature at which the sheath melts. A combination of mono-component fibers having different melting points may also be useful for this purpose. In some embodiments, at least a portion of the fibrous layer is elastic and includes any of the elastic materials described above.

Nonwoven webs useful as the fibrous layer in the composite elastic material and process according to the present disclosure are typically bonded (e.g., point bonded or continuously bonded) before being bonded to the elastic layer and the structured film layer. Accordingly, the bonded nonwoven can have a bonding pattern distinct from the bonding pattern used for bonding the elastic layer to the structured film layer. Such a distinct bonding pattern can be observed in the areas of the bonded nonwoven that extend beyond the border of the structured film layer or on the surface that is not covered by the elastic film layer and the structured film layer. Point bonding can be carried out with a patterned calender roll or with an ultrasonic horn and a patterned anvil roll. Nonwoven webs can also be randomly bonded by powder bonding, wherein a powdered adhesive is distributed through the web and then activated, usually by heating the web and adhesive with hot air. A spray adhesive may also be applied. Through-air bonding may also be useful when no adhesive is applied when hot air can melt bond some of the fibers together. For example, including a relatively low-melting fiber or a bi-component fiber with components of differing melting points may be useful when through-air bonding nonwovens.

Composite elastic material according to the present disclosure and/or made according the process of the present disclosure are useful, for example, in absorbent articles. Absorbent articles according to the present disclosure include diapers and adult incontinence articles, for example. A schematic, perspective view of one embodiment of an absorbent article 100 according to the present disclosure is shown in FIG. 4. Absorbent article 100 includes a chassis 130 with a topsheet side 132 and a backsheet side 134. The chassis 130 also has first and second opposing longitudinal edges 136 and 138 extending from a rear waist region 142 to an opposing front waist region 144. The longitudinal direction of the absorbent article 100 refers to the direction extending between the rear waist region 142 and the front waist region 144. Therefore, the term “longitudinal” refers to the length of the absorbent article 100, for example, when it is in an open configuration.

In some embodiments of the absorbent article disclosed herein, including the embodiment illustrated in FIG. 4, the absorbent article 100 has ear portions 150 in the rear waist region 142 which comprising the composite elastic material of the present disclosure and/or made by the process disclosed herein. Composite elastic materials useful as ear portions 150 can conveniently be made by a process, for example, in which the elastic layer is stretched in either the cross direction or the machine direction (e.g., using any of the methods described above) when bonded to the structured film layer. For the purposes of the present disclosure, the ear portions are considered part of the chassis 130.

Absorbent articles (e.g., incontinence articles and diapers) according to the present disclosure may have any desired shape such as a rectangular shape, a shape like the letter I, a shape like the letter T, or an hourglass shape. The absorbent article may also be a refastenable pants-style diaper with a portion of the composite elastic material of the present disclosure along each longitudinal edge. In some embodiments, including the embodiment shown in FIG. 4, the composite elastic material is included in separate side panels that are attached to the sandwich of at least topsheet, backsheet, and absorbent core during manufacturing of the absorbent article, for example, to form ear portions 150. In the illustrated embodiment, the absorbent article also comprises an elastic material 149 along at least a portion of first and second longitudinal side edges 136 and 138 to provide leg cuffs.

When the absorbent article 100 shown in FIG. 4 is worn, the ear portions 150 in the rear waist region 142 may be wrapped around the wearer's body to overlap and engage with the front waist region 144. In some embodiments, the structured film layer on the ear portions 150 can be engaged with a target area (not shown) comprising a fibrous material arranged on the backsheet of the front waist region 144. For example, loop tapes such as those disclosed in U.S. Pat. No. 5,389,416 (Mody et al.) EP 0.341.993 (Gorman et al.) and EP 0.539.504 (Becker et al.) may be applied to a target area to provide an exposed fibrous material. In other embodiments, the backsheet 134 comprises a woven or nonwoven fibrous layer which is capable of interacting with the structured film layer. Examples of such backsheets are disclosed, for example, in U.S. Pat. No. 6,190,758 (Stopper) and U.S. Pat. No. 6,075,179 (McCormack et al.). In these embodiments, the structured film layer of the ear portions 150 advantageously may engage with any suitable location on the backsheet, which can be determined by the size of the wearer and the desired fit.

While in the illustrated embodiment, the composite material is included in ear portions 150, in other embodiments, the composite elastic material may be included in a fastening tab attached to the rear waist region 142 of the absorbent article 100. The composite elastic material according to the present disclosure and/or made by the process disclosed herein may also be useful, for example, for disposable articles such as sanitary napkins.

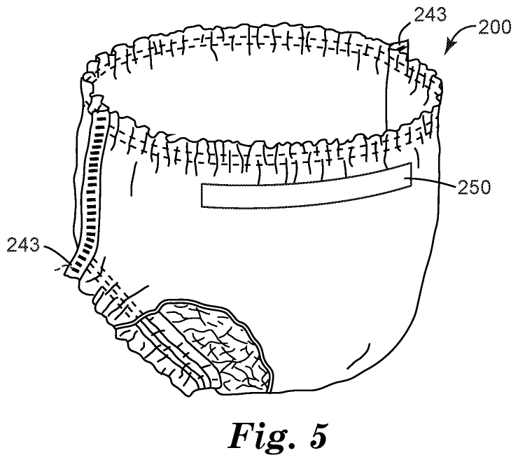

Another embodiment of an absorbent article according to the present disclosure is shown in FIG. 5, which illustrates a pants or shorts style incontinence article 200. Incontinence article 200 may be an infant diaper or adult incontinence article. Like the absorbent article 100 described above, incontinence article 200 includes at least a topsheet, a backsheet, and an absorbent core. In the pants style incontinence article 200, the front waist region and the rear waist regions as well as the leg openings are connected by seams 243. Incontinence article 200 has a composite elastic material 250 of the present disclosure on a portion of the backsheet. Composite elastic material 250—can be useful, for example, for holding the incontinence article in place inside the wearer's clothing. The upstanding male fastening elements (not shown) on the composite elastic material can engage with the fibers in a pair of pants, for example, to ensure the incontinence article stays in place and cannot be seen above the waistline of the pants. Although a variety of constructions may be useful, in the illustrated embodiment, the composite elastic material is shown as a long strip in the rear waist region of the incontinence article.

Following are various, non-limiting embodiments and combinations of embodiments:

In a first embodiment, the present disclosure provides a composite elastic material comprising:

an elastic layer, and

a structured film layer having first and second opposing surfaces, wherein the second surface is bonded to the elastic layer, and wherein the first surface comprises upstanding male fastening elements, wherein the structured film layer is gathered such that the upstanding male fastening elements point in multiple directions.

In a second embodiment, the present disclosure provides the composite elastic material of the first embodiment, wherein the structured film layer has a spacing between gathers of up to five millimeters.

In a third embodiment, the present disclosure provides a stretch-bonded laminate comprising an elastic layer stretch-bonded to a second surface of a structured film layer, wherein a first surface of the structured film layer, opposite the second surface, comprises upstanding male fastening elements.

In a fourth embodiment, the present disclosure provides the stretch-bonded laminate of the third embodiment, wherein the structured film layer is gathered and has a spacing between gathers of up to five millimeters.

In a fifth embodiment, the present disclosure provides the composite elastic material or stretch-bonded laminate of any one of the first to fourth embodiments, wherein the second surface of the structured film layer is discontinuously bonded to the elastic layer at spaced-apart locations, wherein the structured film layer is gathered between the spaced-apart locations.

In a sixth embodiment, the present disclosure provides the composite elastic material or stretch-bonded laminate of any one of the first to fifth embodiments, wherein at least a portion of the structured film layer is microporous.

In a seventh embodiment, the present disclosure provides the composite elastic material or stretch-bonded laminate of any one of the first to sixth embodiments, wherein the structured film comprises a beta-nucleating agent, and/or where at least a portion of the structured film layer comprises beta-spherulites.

In an eighth embodiment, the present disclosure provides the composite elastic material or stretch-bonded laminate of any one of the first to seventh embodiments, wherein the structured film layer, excluding the upstanding male fastening elements, has a thickness in a range from 20 micrometers to 80 micrometers.

In a ninth embodiment, the present disclosure provides the composite elastic material or stretch-bonded laminate of any one of the first to eighth embodiments, wherein at least a portion of the structured film layer has openings therethrough.

In a tenth embodiment, the present disclosure provides the composite elastic material or stretch-bonded laminate of any one of the first to ninth embodiments, wherein at least a portion of the structured film layer excluding the upstanding posts has variations in thickness.

In an eleventh embodiment, the present disclosure provides the composite elastic material or stretch-bonded laminate of any one of the first to tenth embodiments, wherein the elastic layer comprises a fibrous elastic web.

In a twelfth embodiment, the present disclosure provides the composite elastic material or stretch-bonded laminate of any one of the first to eleventh embodiments, wherein the elastic layer comprises a multilayer film.

In a thirteenth embodiment, the present disclosure provides the composite elastic material or stretch-bonded laminate of any one of the first to twelfth embodiments, wherein the elastic layer comprises a plurality of elastic strands.

In a fourteenth embodiment, the present disclosure provides the composite elastic material or stretch-bonded laminate of any one of the first to thirteenth embodiments, wherein the structured film layer is a strip smaller in at least one dimension than the elastic layer.

In a fifteenth embodiment, the present disclosure provides the composite elastic material or stretch-bonded laminate of the fourteenth embodiment, further comprising at least a second strip of the structured film layer bonded to the elastic layer, wherein the second strip is stretch-bonded to the elastic layer and/or wherein the structured film layer is gathered such that the upstanding male fastening elements point in multiple directions.

In a sixteenth embodiment, the present disclosure provides the composite elastic material or stretch-bonded laminate of any one of the first to fifteenth embodiments, further comprising at least one fibrous layer bonded to the elastic layer.

In a seventeenth embodiment, the present disclosure provides the composite elastic material or stretch-bonded laminate of the sixteenth embodiment, wherein the at least one fibrous layer is bonded to the elastic layer on a side opposite the structured film layer.

In an eighteenth embodiment, the present disclosure provides the composite elastic material or stretch-bonded laminate of the sixteenth or seventeenth embodiment, wherein the at least one fibrous layer is disposed between the elastic layer and the second surface of the structured film layer.

In a nineteenth embodiment, the present disclosure provides the composite elastic material or stretch-bonded laminate of any one of the sixteenth to eighteenth embodiments, wherein the at least one fibrous layer comprises a nonwoven material.

In a twentieth embodiment, the present disclosure provides the composite elastic material or stretch-bonded laminate of any one of the first to nineteenth embodiments, wherein the second surface of the structured film layer is bonded to the elastic layer with adhesive.

In a twenty-first embodiment, the present disclosure provides the composite elastic material or stretch-bonded laminate of any one of the first to twentieth embodiments, wherein the second surface of the structured film layer is melt-bonded to the elastic layer.

In a twenty-second embodiment, the present disclosure provides a process for making the composite elastic material or stretch-bonded laminate of any one of the first to fifteenth embodiments, the method comprising:

stretching the elastic layer in a first direction; and

while the elastic layer is stretched, bonding the second surface of the structured film layer to the elastic layer.

In a twenty-third embodiment, the present disclosure provides the process of the twenty-second embodiment, further comprising allowing the elastic layer to relax and the structured film layer to gather to form the composite elastic material.

In a twenty-fourth embodiment, the present disclosure provides the process of the twenty-third embodiment, wherein stretching the elastic layer is carried out in the first direction by differential speed rolls comprising a second roll having a faster speed than a first roll, and wherein allowing the elastic layer to relax is carried out by passing the composite elastic material or stretch-bonded laminate over a third roll having a slower speed than the second roll.

In a twenty-fifth embodiment, the present disclosure provides the process of the twenty-third embodiment, wherein allowing the elastic layer to relax is carried out in a holding box.

In a twenty-sixth embodiment, the present disclosure provides the process of the twenty-third embodiment, wherein allowing the elastic layer to relax is carried out after the composite elastic material or stretch-bonded laminate is incorporated into an article.

In a twenty-seventh embodiment, the present disclosure provides the process of any one of the twenty-second to twenty-sixth embodiments, further comprising unwinding the structured film layer from a roll before bonding it to the elastic layer.

In a twenty-eighth embodiment, the present disclosure provides the process of any one of the twenty-second to twenty-seventh embodiments, wherein bonding comprises adhesive bonding.

In a twenty-ninth embodiment, the present disclosure provides the process of any one of the twenty-second to twenty-eighth embodiments, bonding comprises melt-bonding.

In a thirtieth embodiment, the present disclosure provides the process of the twenty-ninth embodiment, wherein bonding comprises at least one of ultrasonic welding, calendering, or bonding with a heated fluid.

In a thirty-first embodiment, the present disclosure provides the process of the thirtieth embodiment, wherein bond sites are formed by at least one of ultrasonic welding or calendering, and wherein the upstanding male fastening elements are absent in the bond sites.

In a thirty-second embodiment, the present disclosure provides the process of any one of the twenty-second to thirty-first embodiments, further comprising bonding at least one fibrous web to the elastic layer while the elastic layer is stretched.

In a thirty-third embodiment, the present disclosure provides the process of the thirty-second embodiment, wherein the at least one fibrous layer is bonded to the elastic layer on a side opposite the structured film layer.

In a thirty-fourth embodiment, the present disclosure provides the process of the thirty-second or thirty-third embodiment, wherein the at least one fibrous layer is disposed between the elastic layer and the second surface of the structured film layer.

In a thirty-fifth embodiment, the present disclosure provides the process of any one of the thirty-second to thirty-fourth embodiments, wherein the at least one fibrous layer comprises a nonwoven material.

In a thirty-sixth embodiment, the present disclosure provides the process of any one of the twenty-second to thirty-fifth embodiments, wherein the elastic layer is a multilayer film.

In a thirty-seventh embodiment, the present disclosure provides the process of the thirty-sixth embodiment, wherein the multilayer film comprises an elastic core and two opposing less elastic skin layers, and wherein before stretching the elastic layer in the first direction, the method further comprises:

stretching the elastic layer in a direction perpendicular to the first direction to plastically deform the skin layers; and

allowing the elastic layer to relax.

In a thirty-eighth embodiment, the present disclosure provides an absorbent article comprising the composite elastic material or stretch-bonded laminate (f any one of the first to twenty-first embodiments.

Embodiments of the present disclosure have been described above and are further illustrated below by way of the following Examples, which are not to be construed in any way as imposing limitations upon the scope of the present disclosure. On the contrary, it is to be clearly understood that resort may be had to various other embodiments, modifications, and equivalents thereof which, after reading the description herein, may suggest themselves to those skilled in the art without departing from the spirit of the present disclosure and/or the scope of the appended claims.

### EXAMPLES

The following examples are intended to illustrate exemplary embodiments within the scope of this disclosure. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. At the very least, and not as an attempt to limit the application of the doctrine of equivalents to the scope of the claims, each numerical parameter should at least be construed in light of the number of reported significant digits and by applying ordinary rounding techniques.

A box of protective underwear, manufactured by Kimberly Clark, Neeanah, Wis., and sold under the trade designation “DEPEND SILHOUETTE” briefs was obtained from a retail store. While the underwear was in a relaxed state (i.e., with no tension applied), a strip measuring 3.0 centimeters (cm) by 10.2 cm was cut from the elastic waistband. The long direction of the strip with in the extension direction of the elastic. The elastic was extended to 100% extension (20.3 cm) and held at that extension by taping the ends to a table with masking tape.

A strip measuring 2.54 cm by 17.8 cm was cut from the structured film indicated in the Table, below. For Example 1, the structured film was obtained from 3M Company, St. Paul, Minn., under the trade designation “HV-Series” fastener. The structured film had a thickness of 60 micrometers.

For Example 2, the structured film was generally as formed by Example 3 of U.S. Pat. No. 9,358,714 with the modifications that the resin used was polypropylene “5571” impact copolymer from Total Petrochemicals, Port Arthur, Tex., which is reported to have a density of 0.905 g/cc as measured by ASTM D-1505 and a melt flow index of 7 grams per 10 minutes as measured by ASTM 1238. Instead of a post density of 5200 pins per square inch (806 pins per square cm) the structured film had a post density was 3500 pins per square inch (542 pins per square cm). Instead of using a draw ratio of 2:1, the sample was stretched at 70° C. using a draw ratio of 3:1.

For Example 3, the structured film was obtained from 3M Company under the trade designation “CS600” fastener. The structured film had a thickness of 95 micrometers.

A strip of transfer adhesive obtained from 3M Company under the trade designation “3M 1524” medical transfer adhesive measuring the same size as the structured film was applied to the second surface of the structured film, which is the surface opposite the first surface having upstanding male fastening elements. The protective liner was removed from the transfer adhesive.

The structured film was laminated by hand with finger pressure to the elastic while the elastic was being stretched at 100% extension. The stretch bonded laminate (i.e., composite elastic material) was relaxed to a length of 15.2 cm, which is 50% elongation from the initial length, and held at that extension by taping the ends to a table with masking tape. The number of gathers in the final 15.2-cm length were counted and recorded in Table 1, below.

TABLE

| | | Number of | Gathers |
|---|---|---|---|
| Example | Structured Film | Gathers | per cm |
| Example 1 | “HV-Series” | 17 | 1.12 |
| Example 2 | USPN 9358714, Ex. 3 | 32 | 2.10 |
| Example 3 | “CS-600” | 6 | 0.49 |

While the specification has described in detail certain exemplary embodiments, it will be appreciated that those skilled in the art, upon attaining an understanding of the foregoing, may readily conceive of alterations to, variations of, and equivalents to these embodiments. Accordingly, it should be understood that this disclosure is not to be unduly limited to the illustrative embodiments set forth hereinabove. Furthermore, all publications, published patent applications and issued patents referenced herein are incorporated by reference in their entirety to the same extent as if each individual publication or patent was specifically and individually indicated to be incorporated by reference. Various exemplary embodiments have been described. These and other embodiments are within the scope of the following listing of disclosed embodiments.

1. A composite elastic material comprising:
an elastic layer, and
a structured film layer having first and second opposing surfaces, wherein the second surface is bonded to the elastic layer, and wherein the first surface comprises upstanding male fastening elements, wherein the structured film layer is gathered such that the upstanding male fastening elements point in multiple directions, and wherein the structured film layer has at least one of:
a portion that is microporous,
a portion excluding the upstanding posts that has a thickness in a range from 20 micrometers to 80 micrometers,
a portion that has openings therethrough, or
a portion excluding the upstanding posts that has variations in thickness.

2. The composite elastic material of claim 1, wherein the second surface of the structured film layer is discontinuously bonded to the elastic layer at spaced-apart locations, wherein the structured film layer is gathered between the spaced-apart locations.

3. The composite elastic material of claim 1, wherein the second surface of the structured film layer is bonded to the elastic layer with adhesive.

4. The composite elastic material of claim 1, wherein the second surface of the structured film layer is melt-bonded to the elastic layer.

5. The composite elastic material of claim 1, wherein at least a portion of the structured film layer is microporous.

6. The composite elastic material of claim 1,
wherein the structured film layer comprises a beta-nucleating agent;
wherein at least a portion of the structured film layer comprises beta-spherulites; or
wherein the structured film layer comprises a beta-nucleating agent, and at least a portion of the structured film layer comprises beta-spherulites.

7. The composite elastic material of claim 1, wherein the structured film layer, excluding the upstanding male fastening elements, has a thickness in a range from 20 micrometers to 80 micrometers.

8. The composite elastic material of claim 1, wherein the elastic layer comprises at least one of a fibrous elastic web, a multilayer film, or a plurality of elastic strands.

9. The composite elastic material of claim 1, further comprising at least one nonwoven layer bonded to the elastic layer.

10. A process for making the composite elastic material of claim 1, the method comprising:
stretching the elastic layer in a first direction;
while the elastic layer is stretched, bonding the second surface of the structured film layer to the elastic layer; and
allowing the elastic layer to relax and the structured film layer to gather to form the composite elastic material.

11. The process of claim 10, further comprising unwinding the structured film layer from a roll before bonding it to the elastic layer.

12. The process of claim 10, wherein the elastic layer is a multilayer film comprising an elastic core and two opposing skin layers that are less elastic than the elastic core, and wherein before stretching the elastic layer in the first direction, the method further comprises:
stretching the elastic layer in a direction perpendicular to the first direction to plastically deform the skin layers; and
allowing the elastic layer to relax.

13. The process of claim 10, further comprising bonding at least one nonwoven web to the elastic layer while the elastic layer is stretched.

14. An absorbent article comprising the composite elastic material of claim 1.

15. A stretch-bonded laminate comprising an elastic layer stretch-bonded to a second surface of a structured film layer, wherein a first surface of the structured film layer, opposite the second surface, comprises upstanding male fastening elements.

16. The stretch-bonded laminate of claim 15, wherein the structured film layer has at least one of:
a portion that is microporous,
a portion excluding the upstanding posts that has a thickness in a range from 20 micrometers to 80 micrometers,
a portion that has openings therethrough, or
a portion excluding the upstanding posts that has variations in thickness.

17. The stretch-bonded laminate of claim 15, wherein at least a portion of the structured film layer is microporous.

18. The stretch-bonded laminate of claim 15,
wherein the structured film layer comprises a beta-nucleating agent;
wherein at least a portion of the structured film layer comprises beta-spherulites; or
wherein the structured film layer comprises a beta-nucleating agent, and at least a portion of the structured film layer comprises beta-spherulites.

19. The stretch-bonded laminate of claim 15, wherein the structured film layer, excluding the upstanding male fastening elements, has a thickness in a range from 20 micrometers to 80 micrometers.

20. The stretch-bonded laminate of claim 15, further comprising at least one nonwoven layer bonded to the elastic layer.
